(19) United States
(12) Patent Application Publication
(10) Pub. No.: US 2024/0238398 A1
(43) Pub. Date: Jul. 18, 2024

(54) IMMUNOGENIC COMPOSITION

(71) Applicant: GRIFFITH UNIVERSITY, Nathan, Queensland (AU)

(72) Inventors: Michael GOOD, Nathan, Queensland (AU); Hanan AL-NAZAL, Nathan, Queensland (AU); Danielle STANISIC, Nathan, Queensland (AU)

(21) Appl. No.: 18/560,137

(22) PCT Filed: May 11, 2022
(86) PCT No.: PCT/AU2022/050447

§ 371 Date: Nov. 10, 2023

(30) Foreign Application Priority Data
May 11, 2021 (AU) 2021901392

(51) Int. Cl. A61K 39/018; A61K 39/00; A61K 39/002; A61K 39/005; A61K 39/39; A61P 37/04

(52) U.S. Cl. CPC A61K 39/018; A61K 39/002; A61K 39/005; A61K 39/39; A61P 37/04; A61K 2039/515

(57) ABSTRACT

The present disclosure provides immunogenic compositions and methods of inducing an immune response and/or preventing, treating or ameliorating an infection, disease or condition associated with an erythrocytic organism in a first mammal, wherein the erythrocytes are from a second mammal of a species different from the first mammal.

### CROSS-REFERENCE TO RELATED APPLICATIONS

The present application claims priority from Australian Provisional Patent Application No. 2021901392 filed on 11 May 2021, the contents of which are incorporated herein by reference in their entirety.

### TECHNICAL FIELD

The present disclosure relates to the field of vaccines to protect against disease caused by erythrocytic organisms, such as Babesia sp., and methods of using same.

### BACKGROUND

Babesiosis is primarily tick-transmitted but also is rarely transmitted through blood transfusion, organ transplantation, and perinatally. It has a global distribution and causes significant medical, veterinary and economic impacts (Alvarado-Rybak et al., 2016, Schnittger et al., 2012, Krause, 2019). It is caused by protozoan parasites of the genus Babesia, which are closely related to Plasmodium spp. parasites, the causative agents of malaria. Both genera infect red blood cells as part of their life-cycle and it is this erythrocytic stage that is responsible for pathology. More than 100 species of Babesia spp. parasites have been reported, but only a few are able to infect humans. Of these, B. microti is the dominant species in North America whereas B. divergens is the main species in Europe.

Currently, a live, attenuated vaccine produced in splenectomized calves is used to control bovine babesiosis (Callow and Mellors, 1966). This freshly produced vaccine requires refrigeration and has a short shelf-life. There is no vaccine available for human babesiosis, but a subunit vaccine is widely regarded as the only available option. However, research into a subunit vaccine has been challenging. A major challenge for subunit vaccines is that they target merozoite surface antigens to induce antibodies that block invasion and require potent adjuvants to achieve the required titers. A further challenge is the high degree of antigenic diversity within the parasite (Carcy et al., 2006). Accordingly, there remains a clinical need for vaccines against erythrocytic organisms, such as Babesia, that induce significant protective immunity whilst also demonstrating an improved safety profile.

### SUMMARY

The present disclosure is based on the identification of a culture-based liposomal vaccine that can function as a universal vaccine inducing immunity against different Babesia species. Additionally, one challenge in the development of whole erythrocytic parasite vaccines has been the inclusion of the host red blood cell (RBC) membrane in the composition. This is because anti-RBC antibodies can be induced as a result of the blood used to culture the erythrocytic parasite for the vaccine, which represents a clinically significant issue for both human and veterinary vaccines. Accordingly, the immunogenic composition described herein is suitably produced for administration to one mammalian species, such as dogs and cattle, using the blood of a different mammalian species (e.g., human RBCs) to culture parasite material, which may advantageously avoid the production of anti-red blood cell antibodies and haemolytic sequelae in vaccinated animals.

Accordingly, in a first aspect the present disclosure provides an immunogenic composition for administration to a first mammal, said composition comprising erythrocytes and an erythrocytic organism, wherein the erythrocytes are from a second mammal of a species different from the first mammal.

In some examples, the erythrocytic organism is attenuated, inactivated and/or killed.

In certain examples, the erythrocytes have been previously infected with the erythrocytic organism.

In particular examples, the immunogenic composition does not comprise or is substantially free of an adjuvant.

In alternative examples, the immunogenic composition further comprises an adjuvant, such as a lipid-based adjuvant.

Suitably, the erythrocytes are contained in or otherwise associated with a particle. In such examples, the particle can be a lipid-based particle.

In some examples, the erythrocytes are contained in or otherwise associated with a liposome.

In particular examples, the immunogenic composition further comprises a cell targeting ligand.

Suitably, the erythrocytic organism has been chemically attenuated or inactivated. In certain examples, the erythrocytic organism has been chemically attenuated with a DNA binding agent, such as centanamycin, tafuramycin A and any combination thereof.

In other examples, the erythrocytes have been treated to inactivate or kill the erythrocytic organism.

Suitably, the erythrocytes are intact and/or are lysed.

In some examples, the first mammal is a non-human animal. For such examples, the first mammal is suitably canine or bovine.

In particular examples, the second mammal is human.

Suitably, the erythrocytic organism is an intra-erythrocytic organism or an intra-erythrocytic parasite. In some examples, the erythrocytic organism is selected from the group consisting of a Babesia sp., an Anaplasma sp., an Ehrlichia sp., a Trypanosoma sp., a Theileria sp., a Hepatozoon sp., a Mycoplasma sp., a Bartonella sp. and any combination thereof.

Suitably, the erythrocytic organism is or comprises a Babesia sp. In certain examples, the erythrocytic organism is selected from the group consisting of B. bigemina, B. bovis, B. caballi, B. canis, B. divergens, B. rossi, B. microti, B motasi, and any combination thereof. More particularly, the erythrocytic organism can be or comprise a Babesia sp. and an Anaplasma sp. Even more particularly, the erythrocytic organism can be or comprise Babesia divergens.

Suitably, the immunogenic composition provides heterologous protection against an infection, disease or condition associated with one or more other isolates, strains and/or species of the erythrocytic organism. By way of example, the immunogenic composition may provide heterologous protection against an infection, disease or condition associated with one or more other isolates, strains and/or species of Babesia.

Suitably, the immunogenic composition is for use in a method of:
(a) eliciting an immune response in the first mammal; and/or
(b) preventing, treating or ameliorating an infection, disease or condition associated with the erythrocytic organism in the first mammal.

In a second aspect, the present disclosure provides a method of preventing, treating or ameliorating an infection, disease or condition associated with an erythrocytic organism in a first mammal, said method including the step of administering to the first mammal a therapeutically effective amount of the immunogenic composition of the first aspect.

In a third aspect, the present disclosure relates to a method of inducing an immune response in a first mammal, said method including the step of administering to the first mammal, an effective amount of the immunogenic composition of the first aspect.

In a fourth aspect, the present disclosure provides for the use of the immunogenic composition of the first aspect in the manufacture of a medicament for:
(a) eliciting an immune response in the first mammal; and/or
(b) preventing, treating or ameliorating an infection, disease or condition associated with the erythrocytic organism in the first mammal.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein. It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

FIG. 1: In vivo assessment of chemically attenuated B. microti pRBCs. A and B. To assess attenuation of B. microti parasitized red blood cells (pRBCs), BALB/c mice (n=5/group) were injected intravenously with 10⁶ pRBCs treated with 2 μM, 200 nM or 20 nM of Tafuramycin-A (TF-A). Control mice received 10⁶ untreated pRBCs. C and D. The impact of parasite dose on protection induced by a chemically attenuated whole parasite B. microti vaccine. BALB/c mice (n=5/group) were immunized intravenously on days 0, 14 and 28 with 10⁴, 10⁵ or 10⁶ B. microti parasitized red blood cells (pRBCs) that were chemically attenuated with 2 μM of Tafuramycin-A (TF-A). Control mice received 10⁶ TF-A treated normal mouse red blood cells (mRBCs). Mice were challenged with 10⁶ B. microti pRBCs two weeks after the final immunization. (A and C: Parasitemias were monitored by microscopy post challenge. B and D: Mice were assessed for signs of disease and were scored based on defined criteria listed on a clinical scoresheet. Data are expressed as mean±SEM). E. Pre-challenge B. microti-specific antibody levels in mice vaccinated with three doses of 10⁶ B. microti pRBCs or mouse normal red blood cells (mRBCs) treated with TF-A (n=5/group). Hyperimmune serum was derived from mice that had undergone multiple self-resolving B. microti infections.

FIG. 2: The role of immune and accessory cells in immunity induced by chemically attenuated Babesia parasites. A. To assess the role of B cells, C57B1/6 (n=7/group) and μMT (n=4-5/group) mice were immunized intravenously with three doses of 10⁶ B. microti parasitized red blood cells (pRBCs) or mouse normal red blood cells (mRBCs) treated with 2 μM Tafuramycin-A (TF-A). Following vaccination, mice were challenged with 10⁶ B. microti pRBC and parasitemia monitored by microscopy. The experiment with μMT mice was done once. B. To evaluate the role of antibodies, BALB/c mice (n=5/group) were injected intraperitoneally with 0.5 ml of serum on each of days −1, 0 and 1 relative to challenge on day 0. Serum was derived from mice vaccinated with three doses of 10⁶ B. microti pRBCs or mRBCs treated with TF-A, naïve mice or mice that had undergone multiple self-resolving B. microti infections (hyperimmune). Mice were challenged intravenously with 10⁶ B. microti pRBCs. C. To investigate the role of T-cell subsets in protective immunity, CD4+ and/or CD8+ T-cells were depleted from immunized mice prior to challenge. D. To investigate the roles of cytokines/chemokines in vaccine-induced immunity, vaccinated mice (n=5/group) were administered injections of rat Ig, anti-IL-12, anti-IFN-γ or anti-MCP-1 antibodies on days −1, 0 and 1, relative to challenge on day 0. (E) To investigate the role of macrophages, vaccinated mice (5 per group) received 100 μl of a liposome suspension containing chlodronate (5 mg/ml) or PBS on days −1 and 7 relative to challenge on day 0. Mice were challenged intravenously with 10⁶ B. microti pRBCs two weeks after the final vaccine dose. Depletion studies for the chemically attenuated vaccine were performed once. In all experiments, parasitemia was monitored by microscopy. Data are expressed as mean±SEM.

FIG. 3: Protective immunity is induced by a whole parasite B. microti mannosylated liposomal vaccine. A. Comparative protective efficacy of chemically attenuated and liposomal B. microti vaccines. BALB/c mice (n=7/group) were immunized intravenously with three doses of 10⁶ B. microti parasitized red blood cells (pRBCs) treated with 2 μM Tafuramycin-A (TF-A) or subcutaneously with mannosylated liposomes containing 10⁶ B. microti pRBCs. Control mice received empty liposomes or 10⁶ B. microti (killed by freeze/thawing) in PBS. (B) BALB/c mice (n=7/group) were immunized subcutaneously with three inoculations of mannosylated liposomes containing different doses of B. microti pRBCs. Control mice received liposomes containing 2×10⁷ normal mouse red blood cells (mRBCs). C. BALB/c mice (n=7/group) were immunized subcutaneously with three doses of fresh or lyophilized mannosylated liposomes containing 10⁷ B. microti pRBCs. Control mice received fresh liposomes containing 10⁷ mRBCs. Mice were challenged intravenously with 10⁶ B. microti pRBCs two weeks after the final vaccine dose. Parasitemia was monitored by microscopy. D, E. Mice vaccinated with 3 doses of 10⁷ B. microti lyophilized vaccine were challenged one month and three months post challenge. Data are expressed as mean±SEM.

FIG. 4: Immune parameters following whole parasite Babesia liposomal vaccination. A, B. Activation of CD4″ and CD8″ T cells in the peripheral blood seven days after the third immunization with lyophilized mannosylated and fresh liposomes containing 10⁷ B. microti pRBCs. Control mice received either mannosylated fresh liposomes containing 10⁷ mRBCs. C. Splenocyte proliferative responses to different stimulants in mice (n=3/group) immunized with lyophilized mannosylated liposomes containing 10⁷ B. microti parasitized red blood cells (pRBCs). Proliferation was estimated by ³[H]-thymidine incorporation and measured as corrected counts per minute (CPM). Splenocytes from each mouse were tested in triplicate for each stimulant. D. Pre-challenge, B. microti-specific antibody levels were assessed in mice (n=7/group) vaccinated with three doses of mannosylated lyophilized liposomes containing 10⁷ B. microti parasitized red blood cells (pRBCs) or liposomes containing 10⁷ mouse normal red blood cells (mRBCs). Hyperimmune serum was derived from mice that had undergone multiple self-resolving B. microti infections. E. To investigate the role of the spleen in vaccine-mediated immunity, sham-splenectomized and splenectomized mice were vaccinated with three doses of lyophilized mannosylated liposomes containing 10⁷ B. microti pRBCs. Mice were challenged intravenously with 10⁶ B. microti pRBCs two weeks after the final vaccine dose. Parasitemia was monitored by microscopy. F. Protection against heterologous challenge with 10⁶ B. microti pRBC was assessed in BALB/c mice (n=7/group) following vaccination with three doses of lyophilized mannosylated liposomes containing 10⁷ B. divergens parasitized red blood cells (pRBCs). Control mice received mannosylated liposomes containing normal human red blood cells (hRBC). Parasitemia was monitored by microscopy. Data are expressed as mean±SEM. G Splenocyte proliferative responses to different stimulants in mice (n=3/group) immunized with lyophilized B. divergens liposomes. Proliferation was estimated by 3[H]-thymidine incorporation and measured as corrected counts per minute (CPM). Splenocytes from each mouse were tested in triplicate for each stimulant. Data are expressed as mean±SEM. Where applicable, data were analysed by two-way ANOVA, followed by Tukey's multiple comparison test. * p<0.05, ** p<0.01, p<0.0001.

FIG. 5: Assessment of chemically attenuated B. microti pRBCs in BALB/c and SCID mice. Related to FIGS. 1 and 2. BALB/c and SCID mice (n=5/group) were injected intravenously with 10⁶ B. microti parasitized red blood cells (pRBCs) that were chemically attenuated with 2 μM of Tafuramycin-A (TF-A). Control mice received pRBCs that were not treated with TF-A. A. Parasitemia was monitored by microscopy post challenge. B. Mice were assessed for signs of disease and were scored based on defined criteria listed on a clinical scoresheet. Data are expressed as mean±SEM.

FIG. 6: The protective efficacy of different dosing regimens of chemically attenuated B. microti pRBCs in mice. Related to FIGS. 1 and 2. BALB/c mice (n=5/group) were immunized intravenously with A. three, B. two, or C. one dose of 10⁶ chemically attenuated B. microti parasitized red blood cells (pRBCs) or mouse normal red blood cells (mRBCs). Two weeks after the final vaccine dose, mice were challenged intravenously with 10⁶ B. microti pRBCs. 1^{st} column: Parasitemia was monitored by microscopy post challenge. 2^{nd} column: Mice were assessed for signs of disease and were scored based on defined criteria listed on a clinical scoresheet. 3^{rd} column: Hemoglobin levels in mice were assessed using an Hb201+ analyser. Data are expressed as mean±SEM.

FIG. 7: Long-lived protection is induced by a chemically attenuated whole parasite B. microti vaccine. Related to FIGS. 1 and 2. BALB/c mice (n=5/group) were immunized intravenously three times with 10⁶ B. microti parasitized red blood cells (pRBCs) or mouse normal red blood cells (mRBCs) treated with 2 μM of Tafuramycin-A (TF-A). Mice were challenged intravenously with 10⁶ B. microti pRBCs A. three or B. six months after the final vaccine dose. Parasitemia was measured by microscopy following challenge. Data are expressed as mean±SEM.

FIG. 8: Vaccination with a chemically attenuated B. microti vaccine induces parasite-specific cellular immune responses. Related to FIGS. 1 and 2. Pre-challenge cellular immune responses were assessed in BALB/c mice (n=3/group) immunized intravenously three times with 10⁶ B. microti parasitized red blood cells (pRBCs) or mouse normal red blood cells (mRBCs) treated with Tafuramycin-A (TF-A). A, B, C, D. Parasite-specific cytokine/chemokine production in culture supernatants of the splenocyte proliferation assay were collected after 54 hours and used in cytometric bead arrays. Samples were acquired on a LSR Fortessa flow cytometer and data were analyzed using BD FCAP Array software V3.0.1. Supernatants were pooled from triplicate wells for each stimulus for each mouse. E. Pre-challenge splenocyte proliferative responses to different stimulants in mice (n=3/group) immunized with TF-A treated 10⁶ B. microti pRBCs or mRBCs. Proliferation was estimated by 3[H]-thymidine incorporation and measured as counts per minute (CPM). Splenocytes from each mouse were tested in triplicate for each stimulant. Data represents mean±SEM. ConA: Concanavalin A.

FIG. 9: A. The structure of the Babesia liposome vaccine. Related to FIGS. 3 and 4. The liposomal vaccine was prepared using the thin-film hydration method with the addition of a mannosylated lipid core peptide (‘F3’), as described (Giddam et al, 2016). The liposomes consisted of F3, DPPC, DDAB and cholesterol in a ratio of 10:5:2:1. The liposomes contained parasitized red blood cells (pRBC) killed by freeze/thawing. B. The gating strategy for assessing CD4⁺ and CD8⁺ T cell activation in mice vaccinated with a B. microti liposomal vaccine. Related to FIG. 4. Activation of CD4⁺ and CD8+ T cells was measured in the peripheral blood seven days after the third immunization with mannosylated lyophilized or fresh liposomes containing 10⁷ B. microti pRBCs. Control mice received mannosylated fresh liposomes containing 10⁷ mouse normal red blood cells (mRBCs). Following the surface staining of cells with the relevant anti-mouse antibodies, the samples were acquired on a LSR flow cytometer and the data were analyzed using FlowJo software V10.6.2. (i) The lymphocyte population was initially identified using FSC and SSC. (ii) CD3⁺ T cells were then identified and further differentiated into (iii) CD4⁺ and CD8 T cells. (iv) Activated CD8⁺ T cells were identified as CD3⁺CD8^{lo}CD11a^{hi} (v) Activated CD4⁺ T cells were identified as CD3⁺CD11a^{hi}CD49d^{hi}.

FIG. 10: Parasite-specific cellular immune responses are induced by immunization with a mannosylated liposomal B. microti vaccine. Related to FIGS. 3 and 4. A, B, C, D. Pre-challenge cellular immune responses were assessed in BALB/c mice (n=3/group) immunized three times sub-cutaneously with a mannosylated liposomal vaccine containing 10⁶ B. microti parasitized red blood cells (pRBCs) or PBS. Parasite-specific cytokine/chemokine production in culture supernatants of a splenocyte proliferation assay were collected after 54 hours and used in cytometric bead arrays. Samples were acquired on a LSR Fortessa flow cytometer and data were analyzed using BD FCAP Array software V3.0.1. Splenocytes were stimulated with different doses of B. microti parasitized red blood cells (pRBC) or mouse normal red blood cells (mRBC). Supernatants were pooled from triplicate wells for each stimulus for each mouse. Data are expressed as mean±SEM.

FIG. 11: The protective role of cellular immune responses in mice immunized with a mannosylated liposomal B. microti vaccine. Related to FIGS. 3 and 4. To investigate the role of cellular immune responses, key cellular populations were depleted in BALB/c mice (n=5/group) immunized sub-cutaneously with three doses of mannosylated liposomes containing 10⁷ B. microti parasitized red blood cells (pRBCs) and challenged intravenously with 10⁶ B. microti pRBCs. A. For T cells, vaccinated mice received injections of rat Ig, anti-CD4⁺, CD8⁺ or CD4⁺ and CD8″ antibodies on days −2, −1, 0, 4, 8, 12 and 16, relative to challenge with on day 0. B. For macrophages, vaccinated mice received 100 μl of a liposome suspension containing clodronate (5 mg/ml) or PBS on days −1 and 7 relative to challenge on day 0. C. To assess the role of B cells, C57BL/6 and μMT mice (n=7/group) were immunized sub-cutaneously with three doses of mannosylated liposomes containing 10⁷ B. microti parasitized red blood cells (pRBCs). Control mice received PBS. Mice were challenged intravenously with 10⁶ B. microti pRBCs. Parasitemia was monitored by microscopy and mice were assessed for signs of disease and were scored based on defined criteria listed on a clinical scoresheet. Data are expressed as mean±SEM.

FIG. 12: Xenodiagnosis of infection in vaccinated, splenectomized mice. Related to FIG. 4. To gauge the degree of protection in splenectomized mice that had been vaccinated with three doses of lyophilized mannosylated liposomes containing 10⁷ B. microti pRBCs, we transferred 100 μL of blood from each vaccinated splenectomized mouse to a recipient naïve mouse, six weeks after intravenous challenge with 10⁶ B. microti pRBCs. The recipient mice were then followed weekly for three weeks after receiving the blood to see if any developed a microscopic B. microti infection. Parasitemia was monitored by microscopy after the blood transfer.

### DETAILED DESCRIPTION

### General Techniques and Definitions

Unless specifically defined otherwise, all technical and scientific terms used herein shall be taken to have the same meaning as commonly understood by one of ordinary skill in the art (e.g. in immunology, molecular biology, immunohistochemistry, biochemistry, genomics and pharmacology).

The present disclosure is performed without undue experimentation using, unless otherwise indicated, conventional techniques of molecular biology, microbiology, recombinant DNA technology and immunology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III; DNA Cloning: A Practical Approach, Vols. I and II (D. N. Glover, Second Edition, 1995), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984) and Methods In Enzymology (S. Colowick and N. Kaplan, eds., Academic Press, Inc.), whole of series.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment or embodiment of the present disclosure may be applied mutatis mutandis to any other aspect or embodiment or embodiment of the present disclosure.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise. For example, a reference to “a bacterium” includes a plurality of such bacteria, and a reference to “an antigen” is a reference to one or more antigens.

The term “and/or”, e.g., “X and/or Y” shall be understood to mean either “X and Y” or “X or Y” and shall be taken to provide explicit support for both meanings or for either meaning.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

The disclosure of each patent and scientific document, computer program and algorithm referred to in this specification is incorporated by reference in its entirety.

### Immunogenic Compositions

The present inventors sought to develop a whole parasite vaccine for Babesia that provided protection against infection by heterologous Babesia isolates, strains and species. They demonstrate herein proof-of-principle that a chemically attenuated erythrocytic parasite can induce protection and then developed a strategy to produce a vaccine that mimicked an attenuated erythrocytic parasite and that could be lyophilized without loss of potency.

A major advantage of whole parasite vaccines is that every antigen in the organism is represented in the vaccine, limiting the effects of antigenic variation and polymorphism (Good and Stanisic, 2020). Verma et al. (2020) recently examined 30 of the most immunodominant antigens of B. microti and observed that while some were conserved amongst 38 isolates from the continental United States (Lemieux et al., 2016), eleven demonstrated antigenic diversity with evidence of significant immune pressure, as defined by comparing the ratio of non-synonymous to synonymous mutations. The antigens that they found to be conserved have not been tested as vaccine candidates. In cattle, where more extensive analyses of antigenic diversity have been undertaken, a recent study of 575 blood samples found significant diversity of the merozoite surface antigens, MSA-1, MSA-2b and MSA-2c (Wang et al., 2020).

Accordingly, in a broad form the present disclosure provides an immunogenic composition for administration to a first mammal, said composition comprising erythrocytes and an erythrocytic organism, wherein the erythrocytes are isolated or derived from a second mammal of a species different from the first mammal.

By the first mammal and the second mammal being of different species, this should advantageously avoid the production of allotypic anti-RBC antibodies in the first mammal and by extension the development of an auto-immune haemolytic condition if the erythrocytes were also derived from the first mammal. Such a feature also allows for the utilization of erythrocytes from a particular species of mammal that allow for the in vitro culture and cultivation of the erythrocytic organism therein.

As used herein, the term “immunogenic” will be understood to mean that the composition induces, elicits or generates an immune response. Suitably, the immune response is a protective immune response. By “protective immune response” is meant an immune response that is sufficient to prevent or at least reduce the severity or symptoms of an infection with the erythrocytic organism, such as a Babesia parasite, in the first mammal. As used herein, “elicits an immune response” or “induces an immune response” indicates the ability or potential of the immunogenic composition to elicit or generate an immune response to the erythrocytic organism, upon administration of to the first mammal. As used herein, “immunize” and “immunization” refer to administering the immunogenic composition to elicit or potentiate a protective immune response to the erythrocytic organism.

As used herein “mammal” refers to any mammal capable of infection by the erythrocytic organism or parasite, such as a Babesia parasite, inclusive of humans, bovines, dogs, cats, pigs, deer, horses, donkeys, sheep and goats.

In some examples, the first mammal is a non-human mammal. In this regard, the immunogenic composition may be considered a veterinary composition for use in the treatment, amelioration and/or prevention of infection with the erythrocytic organism in a domesticated mammal.

In particular examples, the first mammal is bovine. As used herein, “bovines” are members of the mammalian sub-family Bovinae and include cattle, buffalo, bison and yaks. Cattle include all breeds and sub-species of the genus Bos, including Bos indicus and Bos taurus and hybrids thereof.

In other examples, the first mammal is canine. As used herein, the term “canine” refers to an animal that is a member of the Canidae family, including dingo, wolf, jackal, fox, coyote, and the domestic dog. Dogs include all breeds and sub-species of the species Canis lupas familiaris or Canis familiaris.

In some examples, the second mammal can be a human.

In specific examples, the first mammal is bovine and/or canine and the second mammal is human.

In alternative examples, the first mammal is human and the second mammal is a non-human animal, such as canine.

### Erythrocytic Organisms

It is contemplated that the erythrocytic organisms may be any as are known in the art. Suitably, the erythrocytic organisms are intra-erythrocytic organisms or intra-erythrocytic parasites.

In some examples, the erythrocytic organisms are apicomplexan or an apicomplexan parasite. Accordingly, the immunogenic composition can include blood-stage intra-erythrocytic parasites, such as merozoites, schizonts, rings or trophozoites, although without limitation thereto. For example, the blood-stage intra-erythrocytic parasites may be purified merozoites or a mixture of isolated merozoites and other blood-stage intra-erythrocytic parasites, such as schizonts, rings and/or trophozoites.

For the purposes of this disclosure, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

In some examples, the erythrocytic organism is attenuated, inactivated and/or killed.

In certain examples, the erythrocytic organism is attenuated. The term “attenuate” means to modify the erythrocytic organisms in a way that they becomes less virulent or pathogenic than they were prior to treatment. More particularly, this means that the erythrocytic organism exhibits a substantially reduced ability to cause a clinical disease while still being able to replicate in the host. Methods of attenuating parasites are known in the art and described, for example, in Good et al., 2013 (Journal of Clinical Investigation). With respect to attenuated erythrocytic organisms, these are suitably included intact in the immunogenic composition.

In particular examples, the erythrocytic organism is inactivated. The term “inactivate” means the erythrocytic organisms have been modified in a way or killed such that they are incapable of reproduction in vivo or in vitro. Various physical and chemical methods of inactivating microorganisms are known in the art, such as being irradiated (e.g., treated with UV, X-ray, electron beam or gamma radiation), heat treated, or chemically treated. In this regard, the immunogenic composition may include a lysate or fraction of the erythrocytic organisms described herein.

In some examples, the erythrocytes are initially infected with the erythrocytic organisms and then treated to kill the erythrocytic organisms. In alternative examples, the erythrocytic organisms are killed prior to being combined or mixed with the erythrocytes, such as lysates thereof, of the second mammal.

Suitably, the erythrocytes have been previously infected with the erythrocytic organism. In this regard, the erythrocytes may be infected with the erythrocytic organism in vitro, ex vivo and/or in vivo.

In particular examples, the immunogenic composition comprises a single species of the erythrocytic organism.

In certain examples, the immunogenic composition comprises two or more species (e.g., 2, 3, 4, 5 etc) of the erythrocytic organism from a single genus.

In other examples, the immunogenic composition comprises two or more species (e.g., 2, 3, 4, 5 etc) of the erythrocytic organism from two or more genera (e.g., 2, 3, 4, 5 etc) thereof. By way of example, the immunogenic composition may comprise one or more species of a first erythrocytic organism from a first genus and one or more species of a second erythrocytic organism from a second genus. In certain examples, the immunogenic comprises a first erythrocytic organism of the genus Babesia and a second erythrocytic organism of the genus Anaplasma.

In some examples, however, the erythrocytic organisms do not include a malarial parasite (i.e., a Plasmodium sp.).

In some examples, the erythrocytic organism is selected from the group consisting of a Babesia sp., an Anaplasma sp., an Ehrlichia sp., a Trypanosoma sp., a Theileria sp., a Hepatozoon sp., a Mycoplasma sp., a Bartonella sp. and any combination thereof. Exemplary Babesia species include B. bigemina, B. bovis, B. caballi, B. canis, B. divergens, B. microti, and B. motasi. Exemplary Anaplasma species include Anaplasma platys, Anaplasma phagocytophila, Anaplasma marginale, and Anaplasma centrale. Exemplary Ehrlichia species include Ehrlichia canis. Exemplary Trypanosoma species include Trypanosoma congolense, Trypanosoma evansi and Trypanosoma cruzi. Exemplary Theileria species include Theileria orientalis and Theileria buffeli. Exemplary Hepatozoon species include Hepatozoon canis and Hepatozoon americanum. Exemplary Mycoplasma species include Mycoplasma haemocanis. Exemplary Bartonella species include Bartonella vinsonii subsp. Berkhoffii, Bartonella henselae, Bartonella bovis and Bartonella chomelii.

In particular examples, the erythrocytic organism is or comprises a Babesia sp. As used herein “Babesia” parasites are any pathogenic protists of the genus “Babesia”. The genus “Babesia” includes pathogenic species such as Babesia bovis, Babesia canis, Babesia bigemina, Babesia divergens, Babesia microti, Babesia caballi, Babesia duncani, Babesia venatorum, Babesia ovis, Babesia ovata, Babesia occultans, Babesia vogeli, Babesia gibsoni and Babesia motasi although without limitation thereto. In one example, the erythrocytic organism is or comprises Babesia divergens.

In another example, the erythrocytic organism is or comprises a Babesia sp., such as Babesia divergens, and an Anaplasma sp.

Accordingly, in a particular form, the present disclosure provides an immunogenic composition for preventing, ameliorating or treating babesiosis in a first mammal, said immunogenic composition comprising erythrocytes and blood-stage Babesia parasites, wherein the erythrocytes are from a second mammal of a species different from the first mammal. Suitably, the erythrocytes have been previously infected with blood-stage Babesia parasites and/or the blood-stage Babesia parasites are attenuated, inactivated or killed.

In another form, the present disclosure provides a method of treating, ameliorating or preventing babesiosis, said method including the step of administering the immunogenic composition disclosed herein to a first mammal to thereby prevent or inhibit Babesia infection or treat an existing Babesia infection in the first mammal.

As used herein, “babesiosis” includes all forms of the disease caused by protozoan protists of the genus Babesia, such as those hereinbefore described.

In examples relevant to babesiosis in bovines, the causative Babesia species are typically Babesia bovis, Babesia bigemina and Babesia divergens. Suitably, for treatment of bovines, the immunogenic composition and method of prophylactic or therapeutic treatment of babesiosis comprises erythrocytes of the second mammal (i.e., a non-bovine mammal, such as a human) infected with said Babesia parasites, such as one or more of Babesia bovis, Babesia bigemina and Babesia divergens. In one particular example, the immunogenic composition and method of prophylactic or therapeutic treatment of babesiosis in bovines comprises human erythrocytes infected with Babesia divergens parasites, such as Babesia divergens parasites that have been attenuated, inactivated or killed.

In examples relevant to babesiosis in canines, the causative Babesia species are typically B. canis, B. vogeli, B. gibsoni, B. rossi and B. vulpes. Suitably, for the treatment of canines, the immunogenic composition and method of prophylactic or therapeutic treatment of babesiosis comprises erythrocytes of the second mammal (i.e., a non-canine mammal, such as a human) infected with said Babesia parasites, such as one or more of B. canis, B. vogeli, B. gibsoni, and B. microti. In particular examples, however, and owing to the heterologous protection conferred by this Babesia species and its ability to be cultivated in human erythrocytes, the immunogenic composition and method of prophylactic or therapeutic treatment of babesiosis in canines comprises erythrocytes of the second mammal, and more particularly human erythrocytes, infected with Babesia divergens parasites, such as Babesia divergens parasites that have been attenuated, inactivated or killed.

Babesiosis can include any non-specific flu-like symptoms, such as fever, chills, sweats, headache, body aches, loss of appetite, nausea, or fatigue and other symptoms such as thrombocytopenia, low or unstable blood pressure and haemolytic anaemia which can lead to jaundice and darkened urine, although without limitation thereto.

One unexpected advantage of the present disclosure is that the immunogenic composition described herein may, upon administration to the first mammal, immunize against infection by heterologous isolates, strains and/or species of the erythrocytic organism. By “heterologous” pathogens means related pathogens that may be different strains or variants of a same or related species. The skilled artisan will further appreciate that the immunogenic composition described herein may, upon administration to the first mammal, immunize against infection by the erythrocytic organism itself and optionally one or more heterologous isolates, strains and/or species thereof.

Accordingly, in some examples, the immunogenic composition provides heterologous protection against an infection, disease or condition associated with one or more other isolates, strains and/or species of Babesia. For example, administration of an immunogenic composition comprising erythrocytes, such as human erythrocytes, and blood-stage Babesia divergens parasites, such as those that are attenuated, inactivated or killed, to the first mammal, can provide heterologous protection against an infection, disease or condition associated with Babesia bovis, Babesia canis, Babesia bigemina and/or Babesia. microti.

Suitably, the immunogenic composition and/or method of prevention, amelioration or treatment of babesiosis are at least partly effective against blood-stage babesiosis. Blood-stage babesiosis is a stage where the Babesia parasite (e.g., merozoite) enters erythrocytes. In the blood stage, the parasite divides several times to produce new merozoites, which leave the red blood cells and travel within the bloodstream to invade new red blood cells. For the sufferer, blood-stage babesiosis is typically characterized by successive waves of fever arising from simultaneous waves of merozoites escaping and infecting red blood cells.

### DNA Binding Agents

In some examples, the erythrocytic organisms are chemically attenuated, such as by treatment with a DNA binding agent.

In some examples, the DNA binding agent is centanamycin or an analog or derivative thereof. Centanamycin is a rationally designed, achiral DNA binding and alkylating agent based on (+)-duocarmycin SA that lacks a stereocenter. Centanamycin binds covalently to adenine-N3 in the DNA sequence motif (A/T)AAA.

In other examples, the DNA binding agent is tafuramycin A or an analog or derivative thereof. Tafuramycin A is a rationally designed, DNA binding and alkylating agent based on duocarmycins that comprises a stereocenter.

By “centanamycin or tafuramycin A analogs or derivatives” is meant any molecule structurally related to centanamycin or, tafuramycin A which exhibits binding to AT-containing nucleotide sequences to thereby induce DNA damage.

Centanamycin, tafuramycin A, analogs or derivatives inclusive of non-chiral, chiral and racemic analogs and derivatives of duocarmycin and CC-1065, non-chiral, chiral and racemic isomers, salts or solvates thereof are also described in WO2002/030894, WO2008/050140, WO2009/064908, Howard et al., 2002 and Purnell et al., 2006, Chavda et al., 2010 and U.S. Pat. No. 6,660,742, which are incorporated by reference herein. Reference is particularly made to seco-iso-cyclopropylfurano[2,3-e]indoline-TMI (TH-III-149 or tafuramycin A) and seco-cyclopropyltetrahydrofurano[2,3-f]quinoline-TMI (TH-III-151 or tafuramycin B) analogs of CC-1065 and the duocarmycins as described in Howard et al., 2002, supra and Purnell et al., 2006, supra.

Achiral seco-hydroxy-aza-CBI-TMI, a seco-cyclopropylpyrido[e]indolone (CPyI) compound, is an example of an analog of centanamcyin as described in Chavda et al., 2010, supra. Racemic and chiral 5-methylfuran analogs of tafuramycin A are described in Purnell et al., 2006, supra.

The chemical formula of tafuramycin A is provided below:

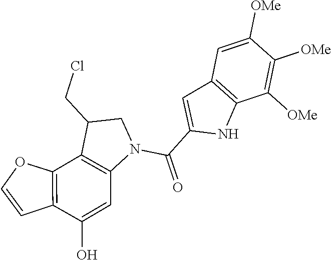 [SMILES: COc1cc2cc(C(=O)N3CC(CCl)c4c3cc(O)c3ccoc43)[nH]c2c(OC)c1OC]

The chemical formula of centanamycin is provided below:

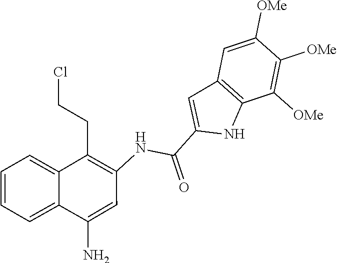 [SMILES: COc1cc2cc(C(=O)Nc3cc(N)c4ccccc4c3CCCl)[nH]c2c(OC)c1OC]

Non-limiting concentrations of the DNA binding agent, including tafuramycin A, centanamycin, or analogs or derivatives thereof, for treatment of red blood cells infected with the erythrocytic organisms described herein, are in the range of about 0.1 to 100 μM. Suitably, the concentration is in the range of about 0.5-50 M, more preferably in the range about 1-40 μM or even more preferably in the range of about 2-20 μM. Particular concentrations include about 0.5 μM, 1.0 μM, 1.5 μM, 2.0 μM, 3 μM, 4 μM, 5.0 μM, 6.0 μM, 7.0 μM, 8.0 μM, 9.0 μM, 10 UM, 11 μM, 12 μM, 13 μM, 14 μM, 15 μM, 16 μM, 17 M, 18 μM, 19 μM, 20 μM, 25 μM and 30 μM, or in any range therebetween.

Treatment duration of the DNA binding agent may be in the range 1 minute to 12 hours, preferably 10 minutes to 4 hours or more preferably about 0.1, 0.2, 03, 0.4, 0.5, 0.6, 0.7, 0.8, 0.9, 1.0, 1.5 or 2 hours, or any range therebetween.

The DNA binding agent-treated erythrocytic organisms may be used to infect red blood cells or erythrocytes of the second mammal, which are then used to prepare the immunogenic composition. Alternatively, red blood cells infected with erythrocytic organisms may be treated with the DNA binding agent and then used to prepare the immunogenic composition.

In some examples, the erythrocytes in the immunogenic composition may be administered as intact cells or as a lysate.

The erythrocytes may be obtained from blood of the second mammal infected with the erythrocytic organism prior to DNA binding agent treatment. Alternatively, to produce parasitized or infected erythrocytes or RBCs (i.e., pRBC) in vitro, non-infected red blood cells may be obtained from the second mammal and then infected in vitro with the erythrocytic organisms pre-treated with the DNA binding agent, or with untreated erythrocytic organisms so that the pRBC are thereafter treated with the DNA binding agent.

It will be appreciated from the foregoing that one example of the present disclosure relates to in vitro treatment of isolated or purified blood-stage intra-erythrocytic parasites, such as merozoites or red blood cells infected with blood-stage parasites (e.g. merozoites, schizonts, rings or trophozoites, although without limitation thereto), with a DNA binding agent (e.g., centanamycin, tafuramycin A or an analog or derivative of centanamycin or tafuramycin A). This treatment is effective to chemically attenuate the blood-stage intra-erythrocytic parasites (e.g., merozoites, schizonts, rings or trophozoites, although without limitation thereto) without killing the parasite, such as by inhibiting parasite replication. Typically, the attenuated blood-stage intra-erythrocytic parasites are not capable of proliferation, or are capable of only limited proliferation, following attenuation by treatment with the DNA binding agent.

Suitably, the dose of the erythrocytic organisms (i.e., such as in infected erythrocytes or pRBCs) is capable of eliciting an immune response to subsequent infection by said erythrocytic organism. In some examples, the immune response is characterised by inducing a T cell response and, optionally, inducing B cells to produce detectable levels, or only low levels, of antibodies. More particularly, the immunogenic composition may elicit an immune response that is characterized as a CD4+ T cell mediated response (including solely CD4+ T cell-mediated responses and mixed CD4+ and CD8+ T cell-mediated responses), possibly with little or no antibody or B cell mediated response. Suitably, the immunogenic composition immunizes the first mammal to prevent, inhibit or otherwise protect the first mammal against subsequent infection with the erythrocytic organism.

In some examples, a single dose of the immunogenic composition prevents, inhibits or otherwise protects the animal against subsequent infection with the erythrocytic organism. In other examples, two or more doses (e.g., 2, 3, 4, 5 etc doses) of the immunogenic composition prevents, inhibits or otherwise protects the animal against subsequent infection with the erythrocytic organism. In one particular example, two doses of the immunogenic composition prevents, inhibits or otherwise protects the animal against subsequent infection with the erythrocytic organism. In another specific example, three doses of the immunogenic composition prevents, inhibits or otherwise protects the animal against subsequent infection with the erythrocytic organism.

A typical dose of infected erythrocytes or pRBC in the immunogenic composition can be, or equivalent to, about 10² to about 10¹² pRBC. In some examples, the immunogenic composition comprises a dose of infected erythrocytes or pRBC suitable for administration to the first mammal that is no more than, or equivalent to no more than, about 10¹¹ pRBC, such as including 10¹⁰, 10⁹, 10⁸, 10⁷, 10⁶, 10⁵, 10⁴, 10³, 10², or 10¹ pRBC or any range between any of these values.

A typical dose of the erythrocytic organism suitable for administration to the first mammal is in the range of about 10² to about 10¹² erythrocytic organisms, such as including about 10², 10³, 10⁴, 10⁵, 10⁶, 10⁷, 10⁸, 10⁹, 10¹⁰, 10¹¹, or 10¹² erythrocytic organisms or any range between any of these values.

### Formulation

Suitably, the immunogenic composition comprises erythrocytes that are lysed and/or are intact. Accordingly, in examples where the erythrocytes have been lysed, the immunogenic composition may include one or more erythrocyte or RBC components, including cell membranes and fragments, membrane proteins, cytosolic components and soluble proteins thereof. In some examples, the erythrocytes of the immunogenic composition are lysed or substantially lysed. In alternative examples, the erythrocytes of the immunogenic composition are intact or substantially intact.

In some examples, the immunogenic composition does not include or is substantially free of an adjuvant. For such examples, the immunogenic composition suitably includes erythrocytes that are intact and contain erythrocytic organisms that have been attenuated, such as chemically attenuated with a DNA-binding agent. Furthermore, such examples may or may not be provided in particulate form, such as in the form of a lipid vesicle or liposome.

By “substantially free of an adjuvant” means that the immunogenic composition suitably contains less than 1.0% by weight, more particularly less than 0.1% by weight, even more particularly less than 0.01% by weight, or yet even more particularly less than 0.001% by weight of an adjuvant, such as those described herein.

In alternative examples, the immunogenic compositions disclosed herein may further comprise an adjuvant. For such examples, the immunogenic composition suitably includes erythrocytes that have been lysed and erythrocytic organisms that have been killed. In some examples, however, an immunogenic composition that includes erythrocytes that are intact and contains erythrocytic organisms that have been attenuated may further comprise an adjuvant.

The term “adjuvant” refers to a compound or mixture that enhances the immune response to an antigen. Antigens may act primarily as a delivery system, primarily as an immune modulator or have features of both. Suitable adjuvants include those suitable for use in mammals, including humans, cattle and dogs.

Examples of known suitable delivery-system type adjuvants that can be used in mammals include, but are not limited to, calcium phosphate; squalane and squalene (or other oils of plant or animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); Bordetella pertussis antigens; tetanus toxoid; diphtheria toxoid; surface active substances such as hexadecylamine, octadecylamine, octadecyl amino acid esters, lysolecithin, dimethyldioctadecylammonium bromide, N,N-dicoctadecyl-N′,N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminium phosphate, alum (e.g., aluminum phosphate, aluminum sulfate or aluminum hydroxide); interleukins such as interleukin 2 and interleukin 12; monokines such as interleukin 1; tumour necrosis factor; interferons such as gamma interferon; immunostimulatory DNA such as CpG DNA, combinations such as saponin-aluminium hydroxide or Quil-A aluminium hydroxide; liposomes (e.g., see International Publication WO2017/070735); ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran alone or with aluminium phosphate; carboxypolymethylene such as Carbopol EMA; acrylic copolymer emulsions such as Neocryl A640 (e.g. U.S. Pat. No. 5,047,238); water in oil emulsifiers such as Montanide ISA 720; oil-in-water emulsions such as MF59 (4.3% w/v squalene, 0.5% w/v polysorbate 80 (Tween 80), 0.5% w/v sorbitan trioleate (Span 85)); poly(D,L-laetide-co-glycolide) (PLG) microparticles or nanoparticles (e.g., the Next Adjuvant or NexaVAC™ System from the NA Vaccine Institute); ballistic particles; ceramic particles; polymeric particles; poliovirus, vaccinia or animal poxvirus proteins; or mixtures thereof.

Accordingly, in some examples, the erythrocytes or the immunogenic composition described herein, are provided in particulate form. A variety of particles may be used for the present disclosure, including but not limited to liposomes, micelles, lipidic particles, ceramic/inorganic particles and polymeric particles. In some particular examples, the immunogenic composition is formulated into a lipid vesicle, and more particularly a liposome. Accordingly, in some examples, the erythrocytes or the immunogenic composition are contained in or otherwise associated with a particle, such as a lipid-based particle or a lipid-based vesicle.

In some examples, the composition does not comprise a saponin-based adjuvant, such as Quil-A®.

In certain examples, the adjuvant comprises at least one vehicle compound or agent. The vehicle compound may be administered as single adjuvant compound, or, more particularly, in combination with one or more further adjuvant compounds.

Suitably, the vehicle compound is a liposome compound, such as a neutral adjuvant compound or formulation, an anionic adjuvant compound or formulation, cationic liposomal vaccine adjuvant, or Stealth liposomes, or JVRS-100 (cationic liposomal DNA complex), or cytokine-containing liposomes, or immunoliposomes containing antibodies to costimulatory molecules, or DRVs (immunoliposomes prepared from dehydration-rehydration vesicles), or MTP-PE liposomes, or Sendai proteoliposomes, or Sendai containing lipid matrices, or Walter Reed liposomes (liposomes containing lipid A adsorbed to aluminium hydroxid), or CAF01 (liposomes plus DDA plus TDB), or CAF04, or CAF09, or CAF10, or AS01 (MPL plus liposome plus QS-21), or AS15 (MPL plus CpG plus QS-21 plus liposome). Moreover the vehicle compound may be formed by a virosome compound (unilamellar liposomal vehicles incorporating virus derived proteins, such as influenza haemagglutinin), e.g. IRIVs (immunopotentiating reconstituted influenza virosomes), or liposomes of lipids plus hemagglutinin. Moreover the vehicle compound may be formed by a virus-like particle (VLP) compound, e.g. Ty particles (Ty-VLPs). Moreover the vehicle compound may be formed by microparticles and/or nanoparticles, such as polymeric microparticles (PLG), or cationic microparticles, or albumin-heparin microparticles, or CRL1005 (block copolymer P1205), or peptomere nanoparticle, or CAP™ (calcium phosphate nanoparticles), or microspheres, or PODDS® (proteinoid microspheres), or nanospheres. Moreover, the vehicle compound may be formed by a protein cochleate compound, especially by stable protein phospholipid-calcium precipitates, such as BIORAL™. Moreover, the vehicle compound may be formed by polymeric particles, ballistic particles or ceramic particles. Also combinations of the different vehicle compounds are envisaged. Accordingly, in some examples, the immunogenic composition further comprises a lipid-containing adjuvant, a lipid-based adjuvant and/or a lipid-derived adjuvant.

Suitably, the immunogenic composition is formulated into a lipid vesicle. As broadly used herein, the lipid vesicle may be a liposome, minicell, multilamellar vesicle, micelle, vacuole or other vesicular structure comprising a lipid bilayer. The erythrocytic organisms, whether intact or lysed and/or attenuated or killed may be located in the intravesicular space or may be displayed on a surface of the lipid vesicle.

The lipid vesicle suitably comprises any lipid or mixture of lipids capable of forming a lipid bilayer structure. These include a phospholipids, sterols inclusive of cholesterol, cholesterol-esters and phytosterols, fatty acids and/or triglycerides. Nonlimiting examples of phospholipids include phosphatidylcholine (PC) (lecithin), phosphatidic acid, phosphatidylethanolamine (PE) (cephalin), phosphatidylglycerol (PG), phosphatidylserine (PS), phosphatidylinositol (PI) and sphingomyelin (SM) or natural or synthetic derivatives thereof. Natural derivatives include egg PC, egg PG, soy bean PC, hydrogenated soy bean PC, soy bean PG, brain PS, sphingolipids, brain SM, galactocerebroside, gangliosides, cerebrosides, cephalin, cardiolipin, and dicetylphosphate. Synthetic derivatives include 1,2-dipalmitoyl-sn-glycero-3-phosphocholine (DPPC), dimethyldioctadecylammonium bromide (DDAB), didecanoylphosphatidylcholine (DDPC), dierucoylphosphatidylcholine (DEPC), dimyristoylphosphatidylcholine (DMPC), distearoylphosphatidylcholine (DSPC), dilaurylphosphatidylcholine (DLPC), palmitoyloleoylphosphatidylcholine (POPC), palmitoylmyristoylphosphatidylcholine (PMPC), palmitoylstearoylphosphatidylcholine (PSPC), dioleoylphosphatidylcholine (DOPC), dioleoylphosphatidylethanolamine (DOPE), dilauroylphosphatidylglycerol (DLPG), distearoylphosphatidylglycerol (DSPG), dimyristoylphosphatidylglycerol (DMPG), dipalmitoylphosphatidylglycerol (DPPG), distearoylphosphatidylglycerol (DSPG), dioleoylphosphatidylglycerol (DOPG), palmitoyloleoylphosphatidylglycerol (POPG), dimyristoylphosphatidic acid (DMPA), dipalmitoylphosphatidic acid (DPPA), distearoylphosphatidic acid (DSPA), dimyristoylphosphatidylethanolamine (DMPE), dipalmitoylphosphatidylethanolamine (DPPE), dimyristoylphosphatidylserine (DMPS), dipalmitoylphosphatidylserine (DPPS), distearoylphosphatidylethanolamine (DSPE), dioleoylphosphatidylethanolamine (DOPE) dioleoylphosphatidylserine (DOPS), dipalmitoylsphingomyelin (DPSM) and distearoylsphingomyelin (DSSM). The phospholipid can also be a derivative or analogue of any of the above phospholipids.

In one example, the lipid vesicle is a liposome.

In some examples of the present disclosure, the volume-weighted diameter of freshly prepared liposomes is between about 10 μm and about 100 μm (e.g., 10, 15, 20, 25, 30, 35, 40, 45, 50, 55, 60, 65, 70, 75, 80, 85, 90, 95, 100 μm or any range therein), about 20 μm and about 80 μm, and about 20 μm to about 50 μm. The volume weighted diameter of lyophilized liposomes may be between about 25 μm and about 150 μm, 40 μm and about 100 μm, or about 50 μm and 75 μm.

In some examples, the lipid vesicles (e.g., liposomes) are lyophilized, such as to assist with storage and delivery. Methods of lyophilising liposomes are known in the art, as disclosed by Zaman et al. (2016) (the contents of which is included herein by reference). In particular examples, a lyoprotectant, such as hyaluronic acid, γ-cyclodextrin and/or trehalose is used. In such examples, the lyophilised liposomes can be resuspended in any suitable buffer prior to administration. Typically, the lyophilised liposomes are resuspended in PBS prior to administration.

In some examples, the erythrocytes and the erythrocytic organisms of the immunogenic composition, inclusive of lysates or fractions thereof, are contained in or otherwise associated with a liposome.

Accordingly, in certain examples, the vehicle compound or agent is or comprises one or more cationic lipids, as are known in the art. Cationic lipids have been reported to have strong immune-stimulatory adjuvant effect. The cationic lipids of the present disclosure may form liposomes that are optionally mixed with antigen and may contain the cationic lipids alone or in combination with one or more neutral lipids. Suitable cationic lipid species include: 3-[4N-(N,-diguanidino spermidine) carbamoyl] cholesterol (BGSC); 3-[N,N-diguanidinoethyl-aminoethane)-carbamoyl] cholesterol (BGTC); N,N N2N3 Tetra-methyltetrapalmitylspermine (cellfectin); N-t-butyl-N′ tetradecyl-3-tetradecyl-aminopropion-amidine (CLONfectin); dimethyldioctadecyl ammonium bromide (DDAB); 1,2-dimyristyloxypropyl-3-dimethyl-hydroxy ethyl ammonium bromide (DMRIE); 2,3-dioleoyloxy-N-[2(sperminecarboxamido)ethyl]-N,N dimethyl-I-p-ropanaminium trifluorocetate) (DOSPA); 1,3-dioleoyloxy-2-(6 carboxyspermyl)-propyl amide (DOSPER); 4-(2,3-bis-palmitoyloxy-propyl)-1-methyl-iH imidazole (DPIM) N,N,N′,N′-tetramethyl-N,N′-bis(2-hydroxyethyl)-2,3-dioleoyloxy-1,4-butanediammonium iodide) (Tfx-50); N-i-(2,3-dioleoyloxy) propyl-N,N,N-trimethyl ammonium chloride (DOTMA) or other N-(N,N-1-dialkoxy)-alkyl-N,N,N-trisubstituted ammonium surfactants; 1,2 dioleoyl-3-(4′-trimethylammonio) butanol-sn-glycerol (DOBT) or cholesteryl (4′trimethylammonia) butanoate (ChOTB) where the trimethylammonium group is connected via a butanol spacer arm to either the double chain (for DOTB) or cholesteryl group (for ChOTB); DORI (DL-1,2-dioleoyl-3-dimethylaminopropyl-hydroxyethylammonium) or DORIE (DL-1,2-O-dioleoyl-3-dimethylaminopropyl-hydroxyethylammoniu-m) (DORIE) or analogs thereof as disclosed in WO 93/03709; 1,2 dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES) or the cationic lipids disclosed in U.S. patent Ser. No. ,283,185, cholesteryl-3-carboxyl-amido-ethylenetrimethylammonium iodide, I dimethylamino-3-trimethylammonio-DL-2-propyl-cholestery carboxylate iodide, cholesteryl-3-O-carboxyamidoethyleneamine, cholesteryl-3-3-oxysuccinamido ethylenetrimethylammonium iodide, 1-dimethylamino-3-trimethylammonio-DL-2-propyl cholesteryl-3-3-oxysuccinate iodide, 2-(2-trimethylammonio)-ethylmethylamino ethyl cholesteryl-3-3-oxysuccinate iodide, 3-N-(N′,N′-dimethylaminoethane) carbamoyl cholesterol (DC-chol), and 3-N-(polyethyleneimine)-carbamoylcholesterol; 0,0′ dimyristyl-N-lysyl aspartate (DMKE); 0,O′-dimyristyl-N-lysyl-glutamate (DMKD); 1,2 dimyristyloxypropyl-3-dimethyl-hydroxy ethyl ammonium bromide (DMRIE); 1,2-dilauroyl sn-glycero-3-ethylphosphocholine (DLEPC); 1,2-dimyristoyl-sn-glycero-3 ethylphosphocholine (DMEPC); 1,2-dioleoyl-sn-glycero-3-ethylphosphocholine (DOEPC); 1,2-dipalmitoyl-sn-glycero-3-ethylphosphocholine (DPEPC); 1,2-distearoyl-sn-glycero-3 ethylphosphocholine (DSEPC); 1,2-dioleoyl-3-trimethylammoninum propane (DOTAP); dioleoyl dimethylaminopropane (DODAP); 1,2-palmitoyl-3-trimethylammonium propane (DPTAP); 1,2-distearoyl-3-trimethylammonium propane (DSTAP), 1,2-myristoyl-3 trimethylammonium propane (DMTAP); and sodium dodecyl sulfate (SDS). The present disclosure contemplates the use of structural variants and derivatives of the cationic lipids disclosed in this application.

Further to the above, the immunogenic composition may contain other lipids in addition to the cationic lipids. These lipids include, but are not limited to, lyso lipids of which lysophosphatidylcholine (1-oleoyl lysophosphatidylcholine) is an example, cholesterol, or neutral phospholipids including dioleoyl phosphatidyl ethanolamine (DOPE) or dioleoyl phosphatidylcholine (DOPC), as well as various lipophylic surfactants, containing polyethylene glycol moieties, of which Tween-80 and PEG-PE are examples. The immunogenic composition may also contain negatively charged lipids as well as cationic lipids so long as the net charge of the complexes formed is positive and/or the surface of the complex is positively charged. Negatively charged lipids of the present disclosure are those comprising at least one lipid species having a net negative charge at or near physiological pH or combinations of these. Suitable negatively charged lipid species include, but are not limited to, CHEMS (cholesteryl hemisuccinate), NGPE (N-glutaryl phosphatidlylethanolanine), phosphatidyl glycerol and phosphatidic acid or a similar phospholipid analog.

In one example, the immunogenic composition comprises a vehicle agent that comprises 1,2-dipalmitoylsn-glycero-3-phosphocholine (DPPC), dimethyldioctadecylammonium bromide (DDAB) and cholesterol. More particularly, the immunogenic composition can contain DPPC, DDAB and cholesterol in the ratio of about 5:2:1.

Methods for producing liposomes to be used in the production of the immunogenic composition of the present disclosure are known to those of ordinary skill in the art. A review of methodologies of liposome preparation may be found in Liposome Technology (CFC Press New York 1984); Liposomes by Ostro (Marcel Dekker, 1987); Methods Biochem Anal. 33:337-462 (1988) and U.S. Pat. No. 5,283,185. Such methods include freeze-thaw extrusion and sonication. Both unilamellar liposomes (less than about 200 nm in average diameter) and multilamellar liposomes (greater than about 300 nm in average diameter) may be used as starting components to produce the complexes described herein.

By way of example, the lipids of choice (and any organic-soluble bioactive), can be dissolved in an organic solvent, mixed and dried onto the bottom of a glass tube under vacuum. The lipid film is rehydrated using an aqueous buffered solution containing the infected erythrocytes to be encapsulated by gentle swirling. The hydrated lipid vesicles can then be further processed by extrusion, submitted to a series of freeze-thawing cycles or dehydrated and then rehydrated to promote encapsulation of antigenic preparations. Liposomes can then be washed by centrifugation or loaded onto a size exclusion column to remove unentrapped bioactive from the liposome formulation and stored at 4° C. The basic method for liposome preparation is described in more detail in Thierry et al., (1992, Nuc. Acids Res. 20:5691-5698).

In some examples, one or more cell-targeting ligands and/or lipid adjuvants can be at least partially encompassed in the lipid bilayer of the liposome. In such examples, an appropriate amount of the molecule can be included in the liposome preparation. The exact amount of cell-targeting ligand and/or adjuvant will be independently dependent on a range of properties, including but not limited to the affinity of the molecule to bind its target, the concentration of the target, the half-life of the molecule, etc.

In one example, the adjuvant is an aluminium-based adjuvant. For example, aluminium salts (alum) may be used as an adjuvant (e.g., aluminium phosphate, aluminium sulfate or aluminium hydroxide). In one example, the immunogenic compositions disclosed herein comprise aluminium phosphate, aluminium hydroxide or aluminium sulfate as an adjuvant.

Examples of known suitable immune modulatory type adjuvants that can be used in mammals include, but are not limited to, saponin extracts from the bark of the Aquilla tree (QS21, Quil A), TLR4 agonists such as MPL (Monophosphoryl Lipid A), 3DMPL (3-O-deacylated MPL) or GLA-AQ, LT/CT mutants, cytokines such as the various interleukins (e.g., IL-2, IL-12) or GM-CSF, and the like.

Examples of known suitable immune modulatory type adjuvants with both delivery and immune modulatory features that can be used in humans include, but are not limited to, ISCOMS (see, e.g., Sjolander et al. (1998) J. Leukocyte Biol. 64:713; WO 90/03184, WO 96/11711, WO 00/48630, WO 98/36772, WO 00/41720, WO 2006/134423 and WO 2007/026190) or GLA-EM which is a combination of a TLR4 agonist and an oil-in-water emulsion.

The immunogenic compositions disclosed herein may further comprise a surfactant. Suitable surfactants are known in the art and include, but are not limited to, polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer. In one example, the surfactant is polysorbate 80. The final concentration of polysorbate 80 in the composition may be least 0.0001% to 10% or at least 0.001% to 1% or at least 0.01% to 1% polysorbate 80 weight to weight (w/w).

In one example, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another example, the final concentration of the polysorbate 80 in the formulation is 1% polysorbate 80 (w/w).

The immunogenic compositions disclosed herein may further comprise a buffer. The buffer may be any suitable buffer known in the art. For example, the buffer may be a TRIS, acetate, glutamate, lactate, maleate, tartrate, phosphate, citrate, carbonate, glycinate, histidine, glycine, succinate and triethanolamine bufferphosphate buffer. In one example, the buffer is a phosphate buffer. In another example, the buffer is a succinate buffer. In another example, the buffer is a histidine buffer. In another example, the buffer is a citrate buffer.

The buffer may be selected from USP compatible buffers for parenteral use, in particular, when the pharmaceutical formulation is for parenteral use. For example the buffer may be selected from the group consisting of monobasic acids such as acetic, benzoic, gluconic, glyceric and lactic; dibasic acids such as aconitic, adipic, ascorbic, carbonic, glutamic, malic, succinic and tartaric, polybasic acids such as citric and phosphoric; and bases such as ammonia, diethanolamine, glycine, triethanolamine, and TRIS.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, glycols such as propylene glycols or polyethylene glycol, Polysorbate 80 (PS-80), Polysorbate 20 (PS-20), and Poloxamer 188 (P188) are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

The immunogenic compositions disclosed herein may further comprise one or more other antigens of other pathogens and/or parasites, particularly from bacteria and/or viruses, as are known in the art. For example, with respect to canines, this may include antigens from one or more of canine coronavirus, Canine morbillivirus (i.e., canine distemper virus), canine adenovirus, canine parvovirus, parainfluenza virus, Bordetella bronchiseptica, Leptospira interrogans and Rabies lyssavirus. Referring to cattle, this may include antigens from one or more of a Pasteurella sp., a Clostridium sp. (e.g., C. perfringens, C. tetani, C. botulinum, C septicum), Mannheimia haemolytica, Histophilus sumni, bovine rotavirus, bovine coronavirus, Escherichia coli, Salmonella enterica, a Leptospira sp. (e.g., L. borgpetersenii, L interrogans) and Staphylococcus aureus.

It is envisaged that other components such as immunologically or pharmaceutically acceptable carriers, diluents and/or excipients may be included in the immunogenic composition described herein. Typically, these include solid or liquid fillers, diluents or encapsulating substances that may be safely used in systemic administration. Depending upon the particular route of administration, carriers, diluents and/or excipients may be selected from a group including sugars, starches, cellulose and its derivatives, malt, gelatine, talc, calcium sulfate, vegetable oils, synthetic oils, polyols, alginic acid, isotonic saline, pyrogen free water, wetting or emulsifying agents, bulking agents, coatings, binders, fillers, disintegrants, lubricants and pH buffering agents (e.g. phosphate buffers) although without limitation thereto. The immunogenic composition may be administered to an animal in any one or more dosage forms that include tablets, dispersions, suspensions, injectable solutions, syrups, troches, capsules, suppositories, aerosols, transdermal patches and the like.

By “pharmaceutically acceptable carrier, diluent and/or excipient” or “immunologically acceptable carrier, diluent and/or excipient” is meant a solid or liquid filler, diluent or encapsulating substance that can be safely used in topical or systemic administration to an animal, preferably a mammal, including humans, cattle and dogs.

Formulations may be presented as discrete units such as capsules, sachets, functional foods/feeds or tablets each containing a pre-determined amount of one or more therapeutic agents of the present disclosure, as a powder or granules or as a solution or a suspension in an aqueous liquid, a non-aqueous liquid, an oil-in-water emulsion or a water-in-oil liquid emulsion. Such formulations may be prepared by any of the methods of pharmacy but all methods include the step of bringing into association one or more agents as described above with the carrier which constitutes one or more necessary ingredients. In general, the formulations are prepared by uniformly and intimately admixing the agents of the present disclosure with liquid carriers or finely divided solid carriers or both, and then, if necessary, shaping the product into the desired presentation.

Also disclosed herein is a container comprising the immunogenic compositions disclosed herein. Any suitable container known in the art may be used. For example, the container may be selected from the group consisting of a vial, a syringe, an ampoule, a flask, a fermentor, a bioreactor, a bag, a jar, an ampoule, a cartridge and a disposable pen. In one example, the container is a vial, ampoule or a syringe.

The container may be made of glass, metals (e.g., steel, stainless steel, aluminium, etc.) and/or polymers (e.g., thermoplastics, elastomers, thermoplastic-elastomers). The container may be at least partially siliconized.

### Cell Targeting Ligands

In some examples, the immunogenic composition further comprises a cell targeting ligand. To this end, particles or lipid vesicles of the immunogenic composition (e.g., liposomes) can be targeted to receptors on antigen presenting cells (APCs), for example, by placing ligands for cellular receptors of APCs on the surface of the particle (for example, mannosyl moieties or complement proteins such as C3d). For such examples, the immunogenic composition additionally comprises a cell targeting ligand at or on the surface of the particle or lipid vesicle. The cell-targeting ligand facilitates the delivery of the immunogenic composition to an immune cell, such as an APC. In some particular examples, the immune cell is an APC, such as a dendritic cell and/or a macrophage. In some other examples, the immune cell comprises a mannose receptor or a C-lectin type receptor on its cell surface.

Suitably, the cell-targeting ligand comprises a lipid anchor component, a linker component, and an oligosaccharide component.

In some examples, the oligosaccharide component comprises at least one mannosyl oligosaccharide. By way of example, the mannosyl oligosaccharide may comprise 1, 2, 3, 4, 5 or 6 mannose residues.

The lipid anchor component of the cell targeting ligand suitably binds, attaches to or otherwise integrates with at least one layer of the lipid bilayer of a lipid vesicle or liposome of the immunogenic composition. The lipid anchor may comprise, consist, or consist essentially of, at least one lipid or fatty acid chain thereof. Preferably the lipid is a C4-C20 lipid, or more preferably a C12-C18 lipid. By way of an illustrative example, the lipid may be a C16 lipid, such as palmitate. The lipid may be saturated or unsaturated, although preferably saturated.

The targeting moiety may further comprise a linker component. Suitably, the linker or spacer is located or positioned between the lipid anchor and the mannosyl oligosaccharide. The linker or spacer may comprise, consist, or consist essentially of one or more amino acids or peptides. Non-limiting examples of suitable amino acids include lysine and serine. In some examples, the linker or spacer may comprise polyethylene glycol. In certain examples, the spacer or linker may comprise one or more polyether compounds such as polyethylene glycol (PEG). The number of repeat units (O—CH₂—CH₂) may be 2-10 (e.g., 2, 3, 4, 5, 6, 7, 8, 9, 10 or any range therein). In some examples, the linker component comprises six repeat units (O—CH₂—CH₂). Suitably, the linker or spacer may comprise two or more linked units comprising the polyether compounds such as polyethylene glycol (PEG).

In one example, the targeting ligand is a mannosylated lipid core peptide (MLCP). Typically, the MLCP may be of the general form of the targeting ligands described above. An example is schematically shown in FIG. 9. Exemplary MLCP molecules designated F2-F5 are shown in Table 1 below.

TABLE 1

| Code | Oligosaccharide | Linker | Lipid |
|---|---|---|---|
| F2 | mannose | Lys-Lys-Ser-Ser | C16 |
| F3 | mannose | Lys-Lys-Ser-Ser | 2 × C16 |
| F4 | mannose | (PEG6)₂-Ser-Ser | C16 |
| F5 | (mannose)₄ | (Lys)₂-Lys-Ser-Ser | C16 |

In some particular examples in which the immunogenic composition is encapsulated in a lipid vesicle (e.g., a liposome), the cell-targeting ligand is at least partially embedded in the lipid bilayer of the liposome. Suitably, the cell-targeting is at least partially embedded in the outer layer of the lipid bilayer.

In certain examples, the immunogenic composition comprises F3, DPPC, DDAB and cholesterol in a ratio of 10:5:2:1.

### Methods of Production

Related aspects of the present disclosure provide a method of producing an immunogenic composition for administration to a first mammal, including the steps of; (a) providing erythrocytes and an erythrocytic organism, wherein the erythrocytes are from a second mammal of a species different from the first mammal; and (b) optionally attenuating, inactivating and/or killing the erythrocytic organism.

In some examples, the present method further includes the initial step of culturing or generating the erythrocytes infected with the erythrocytic organism.

In certain examples, the present method further includes the further step of formulating the immunogenic composition such that the erythrocytes are contained in or otherwise associated with a particle, such as a lipid-based particle or a lipid-based vesicle, as hereinbefore described. In this regard, the erythrocytes may be contained in or otherwise associated with a liposome. In some examples, the present step may include combining the erythrocytes with a vehicle agent, such as a cationic lipid or a lipid-based adjuvant. Such a method may further comprise combining the erythrocytes, or an associated particle (e.g., liposomes), with a cell targeting ligand.

In some examples, the present method includes lysing the erythrocytes, such as by one or more freeze-thaw cycles. Additionally, the present method may include lyophilising the immunogenic composition.

In view of the foregoing, the immunogenic composition may be conveniently prepared by a person skilled in the art using standard protocols, such as those hereinbefore provided.

In another broad form, the present disclosure provides an immunogenic composition produced by the method described herein.

### Administration

Suitable regimens for the administration of the immunogenic compositions disclosed herein are known in the art. The above compositions may be administered in a manner compatible with the dosage formulation, and in such amount as effective. The dose administered to the first mammal, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner, such as a medical or veterinary practitioner.

In some cases, as little as one dose of the immunogenic composition is needed, but under some circumstances, such as conditions of greater immune deficiency, a second, third or fourth dose may be given. Following an initial vaccination, subjects can receive one or several booster immunizations adequately spaced.

Accordingly, the methods described herein may comprise administering a priming composition of the immunogenic composition, wherein the immunogenic composition stimulates or otherwise enhances an immune response to an erythrocytic organism in a first mammal, and subsequently administering a later booster composition of the immunogenic composition as described herein.

For example, the booster composition may be administered at least 7, 14, 21 or 28 days, at least 1, 2, 3, 4, 5, or 6 months, or at least 1, 2, 3, 4, or 5 years after the priming composition. The priming and booster compositions may be administered by the same or different routes. For example, the priming and booster doses may both be administered—subcutaneously, intramuscularly, intravenously, or intraperitoneally. Alternatively, the priming dose may be administered locally (e.g., mucosally, such as intranasally) to induce mucosal antigen-specific immune cells, and the booster dose administered subcutaneously, intramuscularly, or intravenously to induce systemic antigen-specific immune cells. Suitably, the booster dose is administered intramuscularly.

Optimal amounts of components for a particular vaccine can be ascertained by standard studies involving observation of appropriate immune responses in subjects. For example, the dosage for human vaccination is determined by extrapolation from animal studies to human data. In another example, the dosage is determined empirically.

A typical dose of the immunogenic composition of the present disclosure for injection has a volume of 0.1 mL to 2 mL, more preferably 0.2 mL to 1 mL, even more preferably a volume of about 0.5 mL.

### Methods of Treatment

The immunogenic compositions disclosed herein or the peptides disclosed herein may be for use in a method of preventing, treating or ameliorating an infection, disease or condition associated with an erythrocytic organism in a first mammal. Accordingly, the methods and compositions disclosed herein may have medical applications.

As used herein, “treating”, “treat” or “treatment” refers to a therapeutic intervention that at least partly ameliorates, eliminates or reduces a symptom or pathological sign of an erythrocytic organism-associated infection, disorder or condition, such as babesiosis, after it has begun to develop.

Treatment need not be absolute to be beneficial to the subject. The beneficial effect can be determined using any methods or standards known in the art.

As used herein, “preventing”, “prevent” or “prevention” refers to a course of action initiated prior to infection by, or exposure to, the erythrocytic organism and/or before the onset of a symptom or pathological sign of an erythrocytic organism-associated infection, disorder or condition, so as to prevent infection and/or reduce the symptom or pathological sign. It is to be understood that such preventing need not be absolute to be beneficial to a subject. A “prophylactic” treatment is a treatment administered to a subject who does not exhibit signs of an erythrocytic organism-associated infection, disorder or condition, or exhibits only early signs for the purpose of decreasing the risk of developing a symptom or pathological sign of an erythrocytic organism-associated infection, disease, or condition.

Thus, also disclosed herein is a method of preventing, treating or ameliorating an infection, disease or condition associated with an erythrocytic organism in a first mammal, comprising administering to the first mammal a therapeutically effective amount of the immunogenic composition disclosed herein.

Also disclosed herein is the use of the immunogenic composition disclosed herein in the manufacture of a medicament for preventing, treating or ameliorating an infection, disease or condition associated with the erythrocytic organism.

Further described herein is a method of inducing or eliciting an immune response in a first mammal, the method comprising administering to the first mammal a therapeutically effective amount of the immunogenic composition described herein.

In a related form, the present disclosure provides a method immunizing a first mammal against an infection, disease or condition associated with an erythrocytic organism, comprising administering to the first mammal a therapeutically effective amount of the immunogenic composition disclosed herein.

The term “therapeutically effective amount” describes a quantity of a specified agent, such as immunogenic composition described herein, sufficient to achieve a desired effect in a subject being treated with that agent. For example, this can be the amount of the immunogenic composition, necessary to elicit an immune response in the first mammal, immunize the first mammal against the erythrocytic organism and/or prevent, treat or ameliorat an infection, disease or condition associated with the erythrocytic organism. Suitably, a “therapeutically effective amount” is sufficient to prevent, reduce or eliminate a symptom of an infection with the erythrocytic organism. More particularly, a “therapeutically effective amount” may be an amount sufficient to achieve a desired biological effect, for example an amount that is effective to decrease or prevent disease progression.

Ideally, a therapeutically effective amount of an agent is an amount sufficient to induce the desired result without causing a substantial cytotoxic effect in the subject. The effective amount of an agent useful for reducing, alleviating and/or preventing an infection of the erythrocytic organism will be dependent on the subject being treated, the type and severity of any associated disease, disorder and/or condition (e.g., disease progression), and the manner of administration of the therapeutic composition.

In particular examples, the present methods exclude the step of administering an adjuvant to the animal. This is particularly so for those examples in which the immunogenic composition comprises intact erythrocytes infected with erythrocytic organisms that have been attenuated, such as chemically attenuated.

So that the present disclosure may be described in detail and put into practical effect, reference is made to the following non-limiting Examples.

### EXAMPLES

### Example 1—Pre-Clinical Evaluation of a Whole Parasite Vaccine to Control Babesiosis

### Experimental Model and Subject Details

### Animals and Ethics Statement

Six to eight week old female BALB/c, C57BL/6, SCID and μMT mice were used for this study. Inbred BALB/c, C57BL/6 and SCID mice were obtained from the Animal Resources Centre, Western Australia. μMT mice were originally obtained from the Jackson Laboratory and were maintained at the Griffith University Animal Facility. All animals were housed in the Institute for Glycomics Animal Facility under Physical Containment level 2 (PC2) conditions. All animal procedures were performed in accordance with the Australian Code for the Care and Use of Animals for Scientific Purposes 8th edition (2013) under ethics approval numbers GLY/07/15/AEC, GLY/08/16/AEC, GLY/13/16/AEC, GLY/02/20/AEC and GLY/17/18/AEC.

Babesia microti and B. divergens Parasites

Babesia microti (King strain) was initially obtained from Dr. Peter Rolls at the Tick Fever Research Centre, Queensland Department of Primary Industries at Wacol, Brisbane, Australia. The strain was originally isolated from a vole (Microtus agrestia) in UK and maintained by mouse-mouse passage. It was brought in from London by IA Clark in 1977 to John Curtin School of Medical Research at ANU; and that isolate was originally obtained from Frank Cox at Kings College in 1973. B. microti was propagated by passage in BALB/c mice.

Babesia divergens parasites were kindly provided by Chery Lobo, New York Blood Center, New York. B. divergens parasites were maintained in in vitro culture in human A+ erythrocytes (NIH Blood Transfusion Service) using complete medium (RPMI-1640 with L-Glutamine, 25 mM Hepes and 50 μg/ml hypoxanthine (KD Medical), 10% heat inactivated A+ human serum (Interstate Blood Bank), 7.5% sodium bicarbonate (Life Technologies) and gentamicin (Life Technologies)). The cultures were grown in a 37° C. humidified incubator in a 90% N2, 5% O₂, 5% CO₂ gas mix (Rodriguez et al., 2014).

### Method Details

Chemical Attenuation of B. microti pRBCs

Chemical attenuation of B. microti with Tafuramycin-A (TF-A) was conducted as described previously (Good et al., 2013). Blood from mice infected with B. microti (as a source of pRBCs) and from naïve mice (as a source of mRBCs) were collected into EDTA tubes. TF-A was diluted to a 20 μM final concentration in RPMI-1640 with glutamine and added to a 25 cm² vented tissue culture flask to give a final TF-A concentration of 2 μM, 200 nM or 20 nM as required. One hundred microlitres of blood containing B. microti pRBCs or mRBCs was added into each flask, as required. The B. microti pRBCs or mRBCs were incubated at 37° C. in a 5% CO₂ incubator for 40 minutes, with gentle mixing every ten minutes. The blood was then removed from the flasks and centrifuged at 433 g for 5 minutes. Following removal of the supernatant, the cell pellets were re-suspended in RPMI-1640 with glutamine and incubated at 37° C. in a 5% CO₂ incubator for a further 40 minutes. The cell pellets were washed with PBS for five minutes at 433 g. A cell count was performed using a haemocytometer, the immunizing dose calculated, and the blood was resuspended in the required volume of PBS for injection. A vaccine dose of 10⁶ pRBC was administered to the mice.

### Formulation of Liposomes Containing Babesia Antigens

The liposomes were prepared using the thin-film hydration method as described previously (Giddam et al., 2016). They consisted of 1,2-dipalmitoylsn-glycero-3-phosphocholine (DPPC)(Avanti Polar Lipids), dimethyldioctadecylammonium bromide (DDAB)(Sigma Aldrich), cholesterol (Merck) in the ratio of 5:2:1. The liposomes also included a mannosylated core peptide, designated “F3” (10 μg/vaccine dose) that was synthesised as previously described (Giddam et al., 2016). F3 was dissolved in methanol and all other components were dissolved in chloroform. The solvent mixture was evaporated under vacuum to form a thin lipid film in the glass flask. The thin film was hydrated at 50-55° ° C. with ruptured pRBCs in PBS or ruptured normal human or mouse RBCs.

For lyophilized liposomes, after the thin film was made, it was hydrated using 20 mM PBS (pH 7.2-7.4) containing 1.3 mM trehalose with ruptured pRBCs or normal human or mouse RBCs. The hydrated liposomes were placed into glass vials and snap-frozen on dry ice-acetone mixture for 5 minutes. The vials, with caps loosened, were placed in a freeze-dryer jar which was connected to the freeze dryer (Christ Alpha 1-4 LOC) at −40° C. with a 0.11 mbar vacuum for 18-20 hours. Following removal from the freeze dryer, the lyophilized liposomes were stored at 4° C. until required for immunization. Immediately prior to immunization, the lyophilized liposomes were rehydrated in the required volume of 1× D-PBS.

### Immunization and Challenge of Mice

Mice were immunized intravenously (i.v.) (for the chemically attenuated B. microti vaccine) or subcutaneously (for the liposomal B. microti divergens vaccines) on days 0, 14 and 28 in a volume of 200 μl. When examining different vaccine dosing regimens for the chemically attenuated vaccine, immunizations were administered on only some of these days, depending on the number of doses. Mice were challenged intravenously with 10⁶ B. microti pRBC in a volume of 200 μl in PBS two weeks after the final vaccine dose. For the chemically attenuated vaccine, parasite challenge was also undertaken three or six months after the final vaccine dose. Post-challenge, mice were monitored by Giemsa-stained thin blood films and by measuring weights and hemoglobin (Hemocue201+Analyser). Mice were also monitored using a clinical scoresheet. Mice that showed signs of severe distress, according to the clinical criteria below, or those that experienced >15% weight loss from the time of challenge, were euthanased using CO₂ gas or by cervical dislocation. The major signs of severe distress (leading to euthanasia of the mice), as agreed to by our Animal Ethics Committee, included two of the following for two consecutive days: (i) Extreme pallor/yellow-green urine or blood in the urine; (ii) Severe hunching impairing movement; (iii) Activity: moderately decreased to stationary (unless stimulated)/abnormal movement; (iv) severe ruffling/poor grooming; (iv) Impaired/failure to respond to external stimuli. For scoring, each of these signs counts as 2 points. Minor signs of distress were: (i) Pallor around the ears/yellow-green urine; (ii) Mild to moderate hunching noted only at rest; (iii) Mild to moderately decreased/slight withdrawal; (iv) Mild to moderate ruffling; (v) Mild to moderately slowed. For scoring, each of these signs counts as 1 point.

With the exception of the experiments that required administration of monoclonal antibodies and/or flow cytometry-based monitoring following challenge, all post-challenge measurements were conducted blinded to prevent observer bias.

Lysis of pRBC and nRBC to Assess Role of Red Cell Membranes

Studies were performed to ask whether intact malaria parasites were required to induce protection. Infected blood was centrifuged at 433 g for five minutes. The plasma was removed. An equal volume of MilliQ water was added and incubated for two minutes at room temperature. Next, a volume of 2× PBS was added into the final volume of both the lysed pRBCs and nRBCs to restore osmolality. Experimental mice were then vaccinated i.v. with the formulation.

### Preparation of Splenectomized Mice

BALB/c mice underwent a laparotomy with repair for removal of the spleen. An equal number of mice underwent sham-splenectomies (ie laparotomy without removal of the spleen). Mice received Isoflurane/Buprenorphine as part of pre-operative and post-operative care. After a recovery period of 4-5 weeks, groups of mice were immunized as described above.

### Assessment of CD4+ and CD8⁺ T Cell Activation

Seven days following the third immunization, peripheral blood was collected by a submandibular bleed into 1 ml of 5 mM EDTA. The blood samples were processed as previously described (Raja et al., 2016). The cells were resuspended in an antibody mastermix containing CD3-V450 (clone 17A2), CD4-V500 (clone RM4-5), CD8 PerCP-Cy5.5 (cline 53.6.7), CD11a-FITC (Clone 2D7) and CD49d-PE (clone 9C10) (all antibodies from BD Biosciences) and were incubated on ice in the dark for 20 minutes. Samples were acquired on a LSR Fortessa flow cytometer (BD Biosciences) and data were analysed using FlowJo software V10.6.2 (Tree Star).

Detection of IgG Against Crude B. microti Antigen by ELISA

Ninety-six well Immunoplates (Nunc) were coated with 10 μg/mL of crude B. microti antigen and stored overnight at 4° C. The plates were washed with PBS 0.05% Tween 20 and then blocked with blocking buffer (10% skim milk in PBS/0.05% Tween20) for 90 minutes at 37° C. Following removal of the blocking buffer, two-fold serial dilutions of pre-challenge serum samples were made in 5% skim milk buffer (5% skim milk in PBS Tween 20) with a starting dilution of 1:50. The plates were incubated at 37° C. for 90 minutes and then washed four times with PBS 0.05% Tween20. Next, the plates were incubated for 90 minutes at 37° C. with goat anti-mouse total IgG-HRP conjugated antibody (Bio Rad) at a dilution of 1:3000 in 5% skim milk buffer. Following further washing with PBS 0.05% Tween20, the plates were incubated with tetramethylbenzidine substrate (TMB) (BD Biosciences) for 15 minutes in the dark. The reaction was stopped by adding 1N sulphuric acid to each well before the absorbance was determined at a wavelength of 450 nm using a xMark™ microplate spectrophotometer plate reader (Bio Rad).

### Measurement of Splenocyte Proliferation

To examine the induction of parasite-specific cellular immune responses, splenocyte proliferation assays were undertaken immediately pre-challenge with pRBCs (B. microti and/or B. divergens) at varying concentrations, or normal RBCs from mice or humans at varying concentrations (negative control), complete RPMI medium alone (RPMI supplemented with 10% heat inactivated newborn calf serum, 1% L-glutamine (100×), 1% penicillin streptomycin and 0.1% 2-mercaptoethanol), concanavalin A (ConA) (10 μg/mL) (positive control). Splenocytes were cultured in triplicate wells for 72 h at 37° C. and 5% CO₂. The uptake of 3[H] thymidine (Perkin-Elmer, USA) was used to assess splenocyte proliferation. Cultures were pulsed with 1 μCi of ³[H] thymidine/well for the last 18 h of the 72 h culture period. Plates were stored at −80° C. prior to thawing and harvesting onto glass fibre filter mats (Perkin-Elmer, USA). Radioisotope incorporation was measured by ß-emission spectroscopy using a MicroBeta ß counter (Perkin Elmer, USA) to obtain radioactivity counts per minute (CPM) values. Culture supernatants were removed prior to addition of the radioisotope and frozen at −80° C. for cytokine analysis.

### Detection of Cytokines and Chemokines in Splenocyte Culture Supernatants

Cytokines and chemokines were measured in the thawed culture supernatants using Mouse Th1/Th2/Th17 and/or Mouse inflammation cytometric bead array kits (BD Biosciences) according to manufacturer's instructions, with minor modifications as previously described (Raja et al., 2016). Samples were acquired on a LSR Fortessa flow cytometer (BD Biosciences) and data were analysed using BD FCAP Array software V3.0.1 (BD Biosciences).

### In Vivo Depletion of Cell Populations and Cytokines Chemokines

Following vaccination, mice were depleted of different cell populations and cytokines according to the following protocols. To assess levels of CD4⁺ T-cells, CD8⁺ T-cells and macrophages, spleens removed from vaccinated, depleted, unchallenged mice were analysed on a BD LSR Fortessa flow cytometer as indicated below. Data were analysed using FlowJo software version 10.6.2. In all experiments, a control group of vaccinated mice received an equivalent amount of non-specific Rat Ig antibodies (Sigma Aldrich) according to the same administration schedule.

To deplete CD4+ and CD8⁺ T cells, mice received intraperitoneal (i.p.) injections of 0.250 mg of anti-CD4+(clone GK1.5, Bio X cell) or 0.500 mg of anti-CD8+(clone 53-5.8, Bio X cell) antibodies on days −2, −1, 0, 4, 8 and 12 for the chemically attenuated vaccine and on days −2, −1, 0, 4, 8, 12 and 16 for the liposomal vaccine relative to challenge on day 0. For the chemically attenuated vaccine, depletion was confirmed by staining splenocytes on days 1, 9 and 16 post-challenge with CD3-V450 (clone 17A2), CD4-V500 (clone RM4-5) and CD8-PerCp-Cy5.5 (clone 53.6.7) (all antibodies from BD Biosciences). For the liposomal vaccine, depletion was assessed at these time points and on day 22.

To deplete macrophages, 100 μl of a liposome suspension (Liposoma, Amsterdam) containing clodronate (5 mg/ml) or PBS was administered i.v. on days −1 and 7 relative to challenge on day 0. Depletion was confirmed by staining splenocytes on days 1 and 9 post-challenge with CD11c-FITC (clone HL3), F4/80-PE (clone T45-2342) (both from BD Biosciences) and a Live Dead stain (Invitrogen).

For depletion of cytokines/chemokines, mice received 1 mg of anti-IFN-γ (clone XMG1.2, Bio X cell) or anti-IL-12p40 antibodies (clone C17.8, Bio X cell) i.p. on days −1, 0 and 1 relative to challenge (Pinzon-Charry et al., 2010). For depletion of MCP-1, 200 μg of anti-MCP-1 antibody (clone 2H5, Bio X cell) was administered on days −1, 0 relative to challenge and then every 4 days (Day 4, 8, 12, 16, 20, 24).

### Passive Serum Transfer Studies

Serum was collected and pooled separately from the following donor mice: naïve mice, mice vaccinated with 10⁶ chemically attenuated B. microti pRBC and mice that had recovered from multiple self-resolving B. microti infections (hyperimmune mice) Naïve recipient mice received 500 μl of the appropriate sera on days −1, 0 and 1 intraperitoneally relative to intravenous challenge with 10⁶ B. microti pRBCs on day 0.

### Quantification and Statistical Analysis

### Statistical Analysis

GraphPad Prism software V6 was used for all statistical analyses. When comparing two experimental groups, an un-paired, two-tailed t-test was used. In experiments where more than two groups were compared, ANOVA was used, which was followed by Tukey's multiple comparisons test. The figure legends detail the number of animals used for each experiment and other specific statistical details.

### Results a Whole Parasite Vaccine can Control Babesiosis.

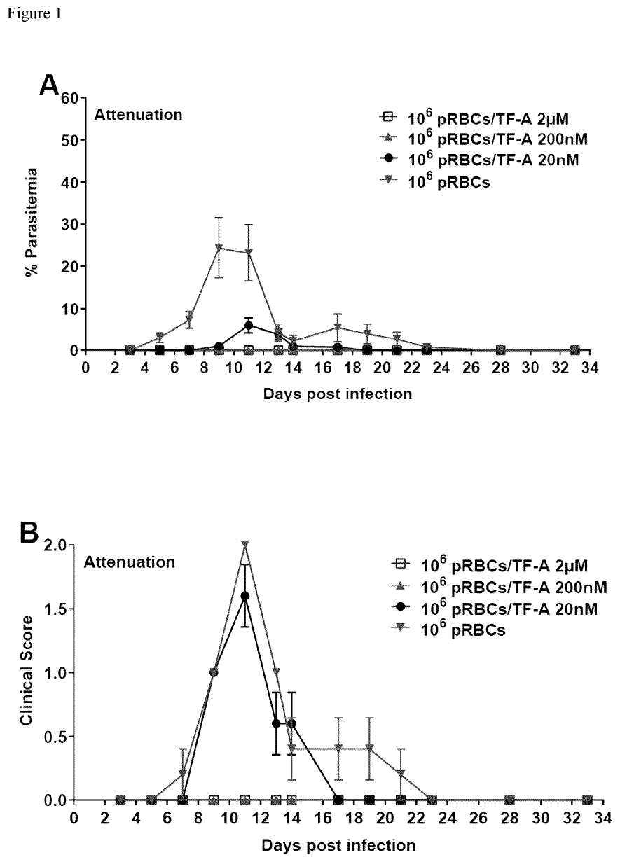

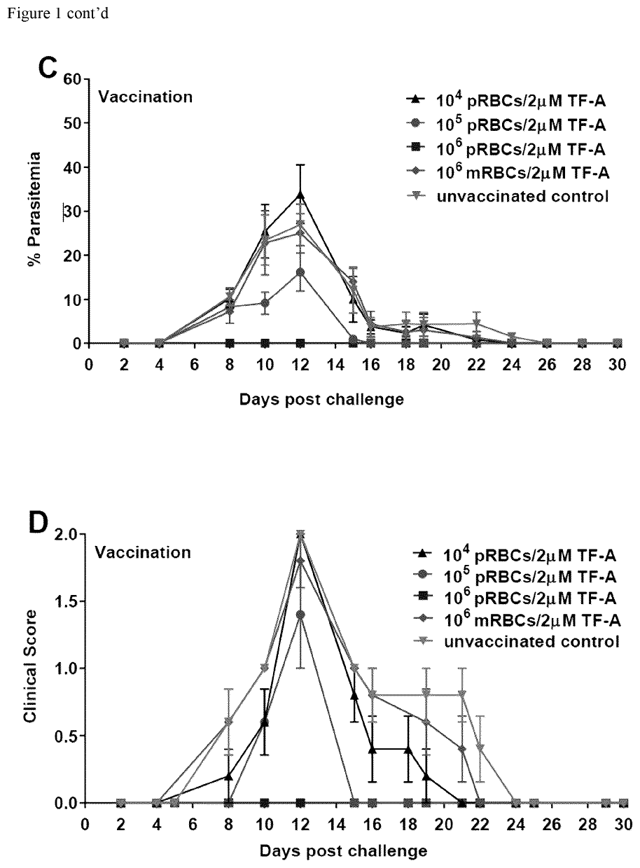

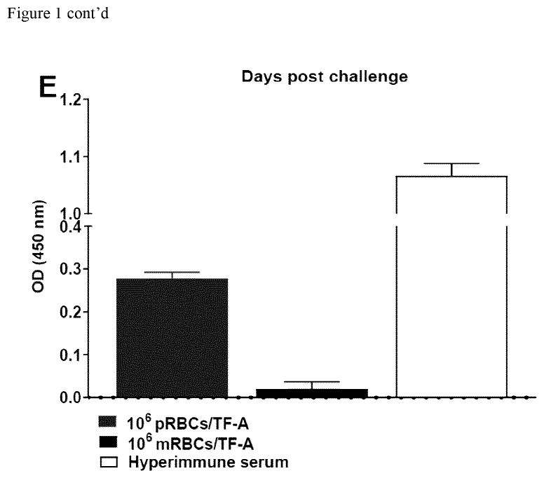

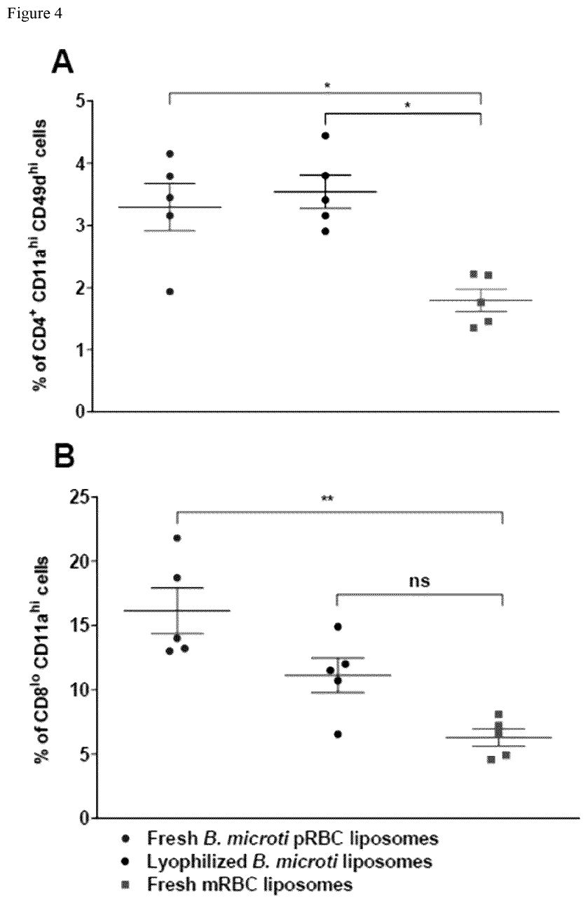

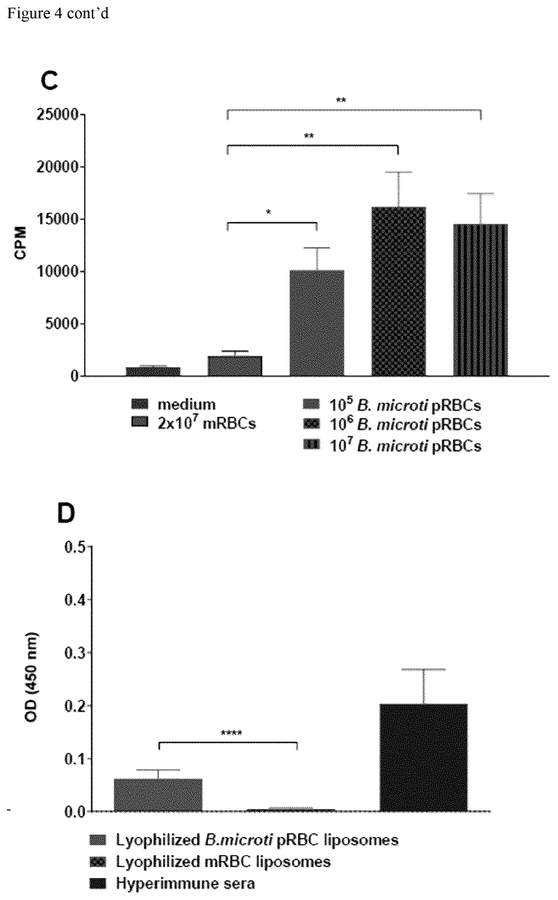

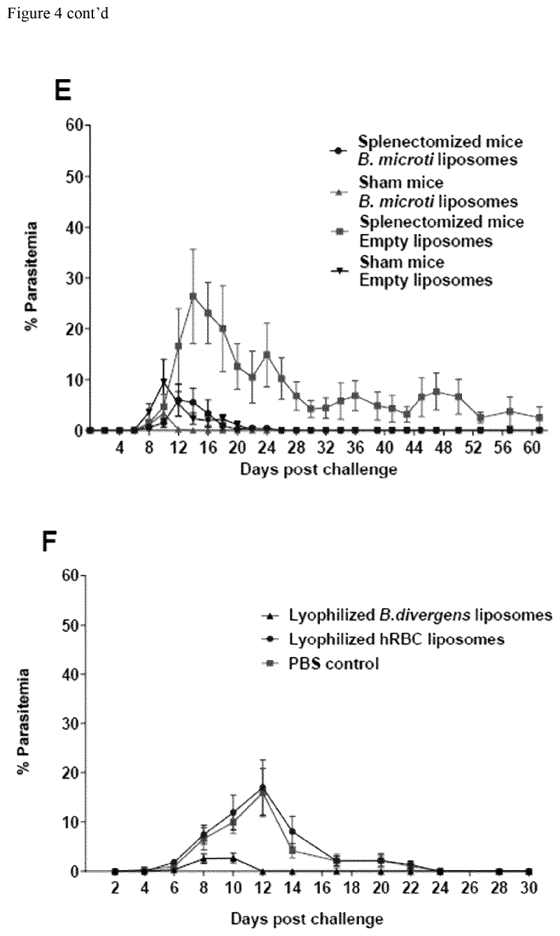

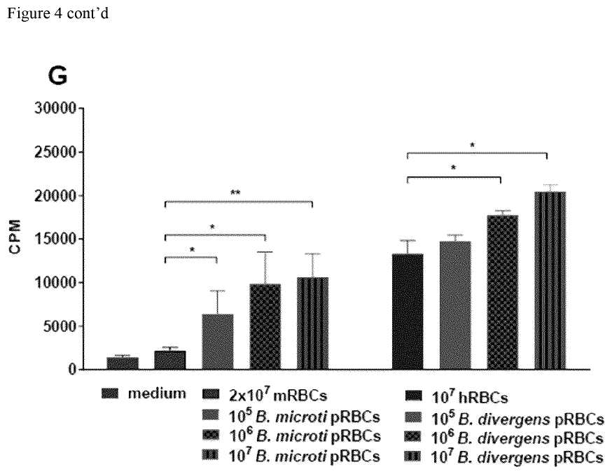

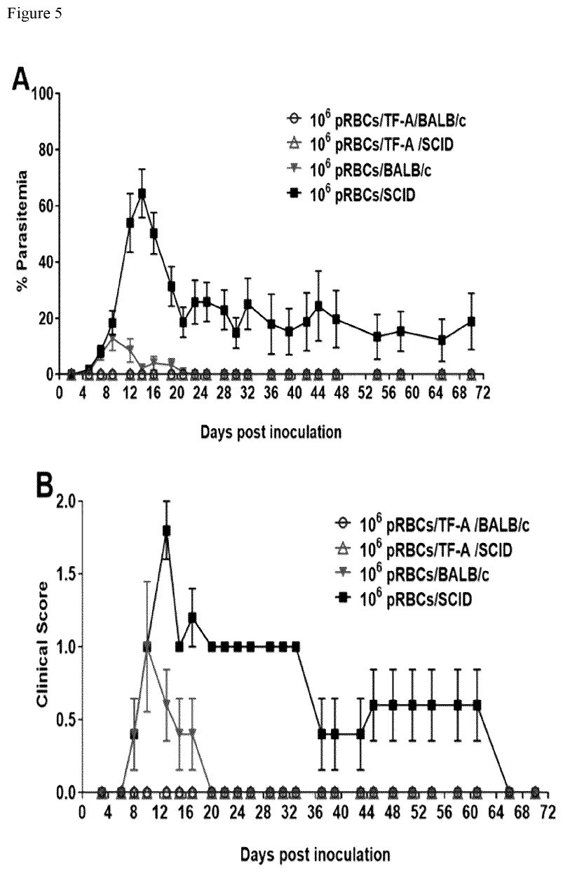

Having previously shown that the seco-cyclopropyl pyrrolo indole analog, Tafuramycin-A (TF-A), could attenuate Plasmodium spp. parasites in vitro and that attenuated parasites could induce immunity (Good et al., 2013, Raja et al., 2016), we asked whether this approach could also be used to induce immunity to Babesia. BALB/c mice that received an intravenous inoculation of 10⁶ B. microti parasitized red blood cells (pRBCs) attenuated with 2 μM or 200 nM, but not 20 nM, of TF-A did not develop microscopically patent parasitemia nor clinical signs of infection (FIGS. 1A and B). 10⁶ B. microti pRBCs, treated with 2 μM TF-A, were then injected intravenously into immunodeficient SCID mice. These mice also remained without signs of clinical disease and were microscopically clear of parasites for 70 days (FIG. 4), further demonstrating that the parasites could be chemically attenuated.

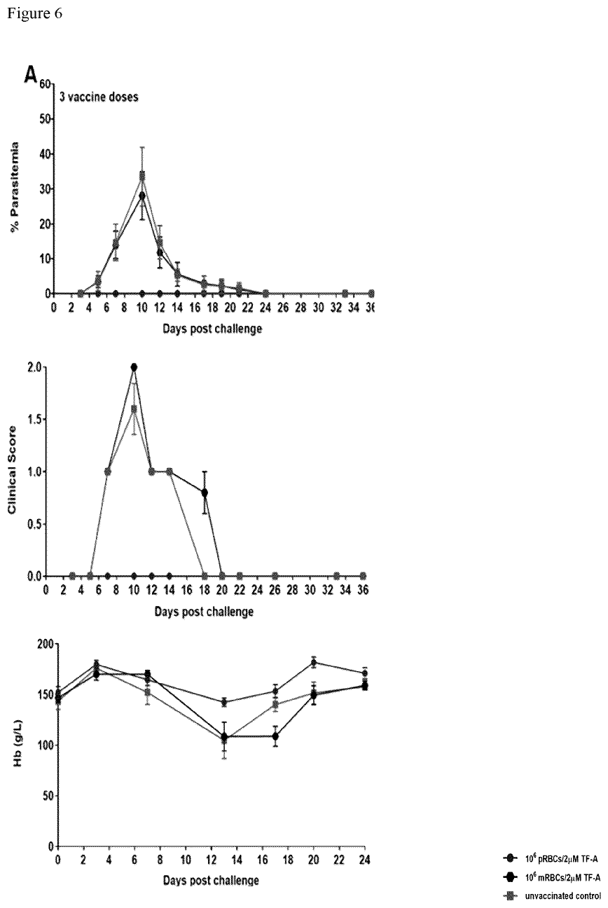

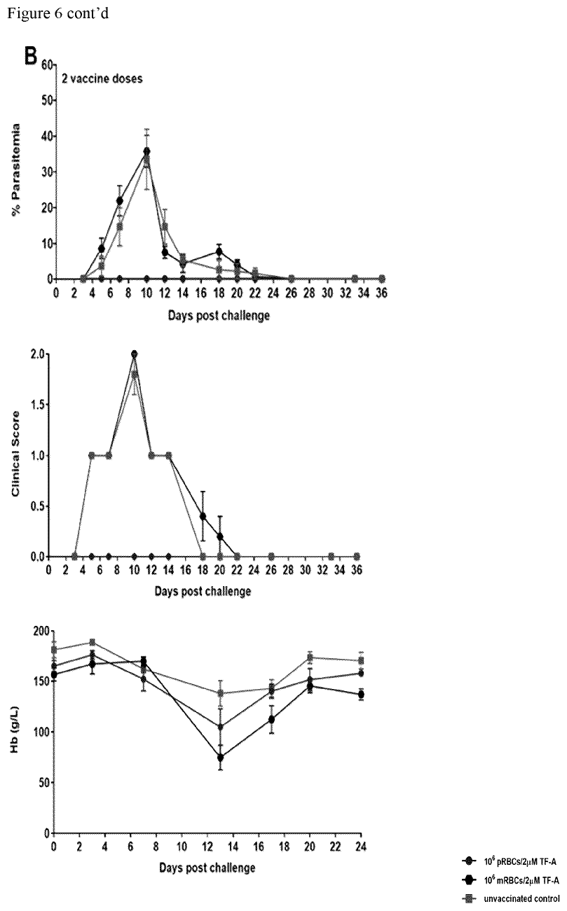

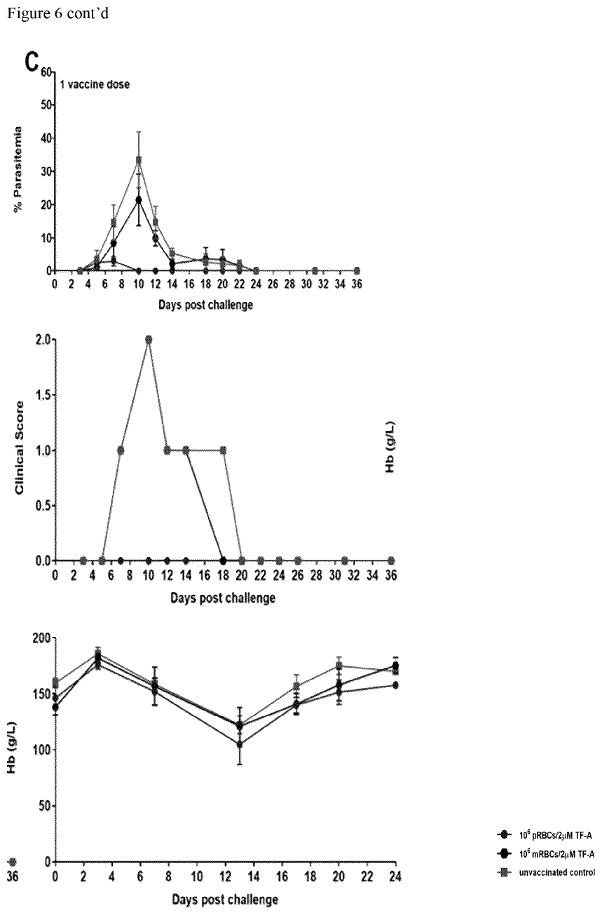

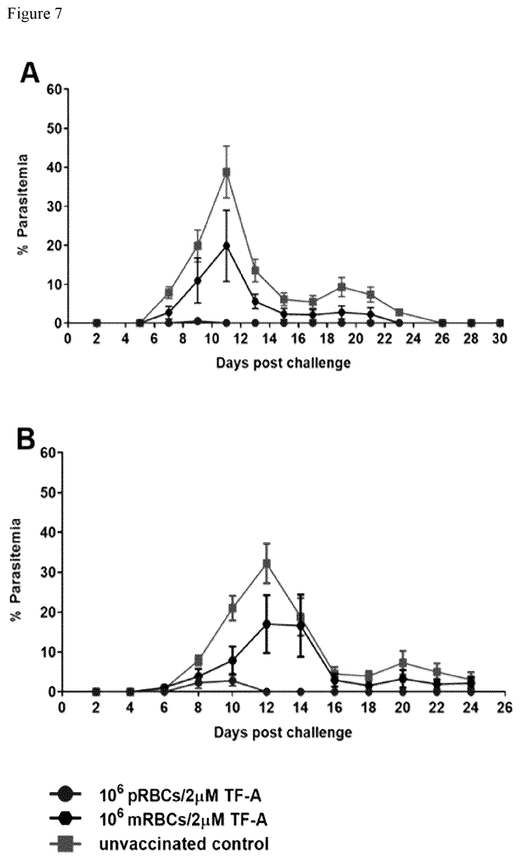

The inventors then tested whether attenuated parasites could induce immunity. Mice were given three doses (each 2 weeks apart) of either 10⁴, 10⁵ or 10⁶ B. microti pRBCs or 10⁶ normal mouse RBCs (mRBCs) treated with 2 μM TF-A. Two weeks after the final vaccination, mice were challenged with 10⁶ homologous parasites. This dose-response study showed that while 10⁶ attenuated pRBC induced strong protective immunity (no parasites detected by microscopy, no clinical score), lower doses (10⁵, 10⁴) were less effective (FIG. 1C, D). Serum taken prior to challenge, showed that they had developed an antibody response to parasite antigens, albeit a much lower response than in mice that had undergone sequential self-resolving infections with unattenuated parasites (FIG. 1E). The optimal number of vaccine doses (using 10⁶ pRBC) was also investigated. Significant protection (reduced parasitemia, low clinical scores, higher haemoglobin levels) was seen following one, two or three doses, although two or three doses gave optimal protection (FIG. 6). However, with just a single immunisation, the peak parasitemia in the vaccinated mice was 2.88%=1.44% versus 21.42%+7.75% and 33.52%+8.41% in the control groups. Vaccine efficacy persisted for at least six months following a 3-dose vaccine regimen (FIG. 7).

It was then asked whether immunity could be induced with the same dose of parasites, but lysed using distilled water (see Materials and Methods). Mice were vaccinated three times with a preparation of 10⁶ lysed pRBC. Following a challenge infection, mice did not demonstrate any protection (data not shown). This did not surprise us as a similar lack of protection was observed when mice were vaccinated with a lysed preparation of malaria parasites, whereas attenuated intact parasites did induce immunity (Good et al., 2013). Those data had indicated that an intact red cell membrane was required to target the parasite to antigen presenting cells (APCs) in the spleen and liver.

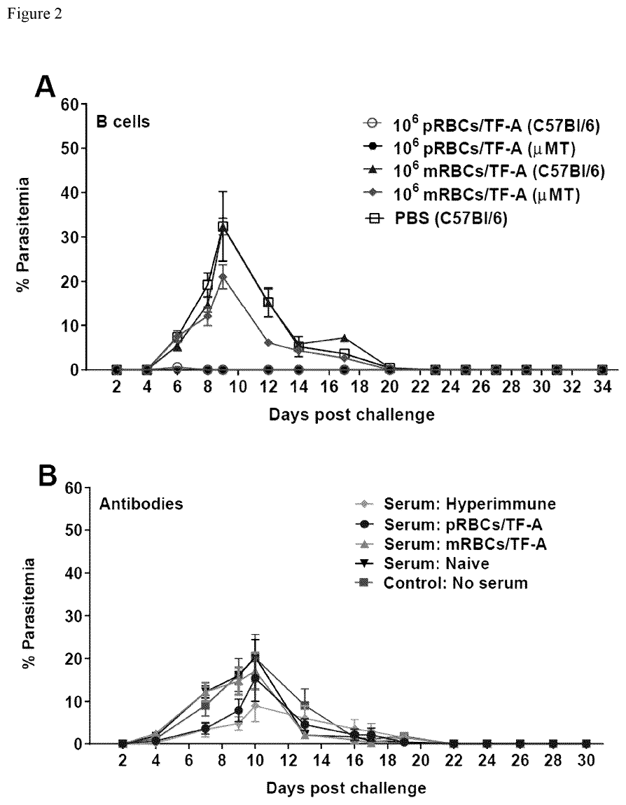

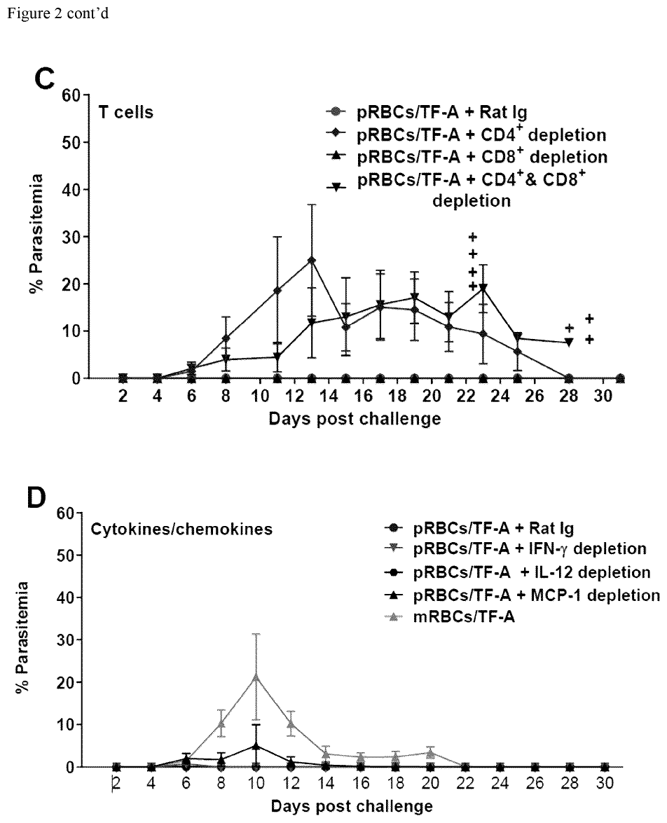

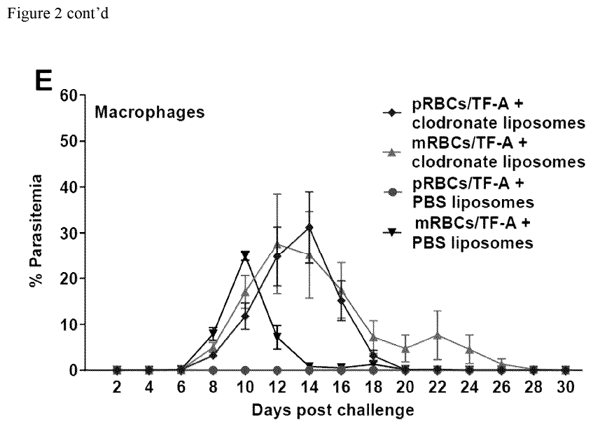

Having observed an antibody response following vaccination, we investigated the role of B-cells in immunity. Genetically modified μMT mice, which lack mature B-cells and antibodies, were immunized with attenuated parasites. However, these mice exhibited equivalent protection to normal mice (FIG. 2A). We explored the role of antibody further by transferring serum from immunized mice to naïve mice prior to challenge. Naïve mice received 500 μL of serum (on each of days −1, 0 and +1 relative to the day of challenge) from mice vaccinated with attenuated parasites, from mice that have recovered from multiple self-resolving infections (‘hyperimmune serum’) and from control mice vaccinated with chemically treated normal mouse red blood cells mRBC. Hyperimmune serum provided partial protection (reduced parasitemia) but serum from mice vaccinated with attenuated parasites did not reduce the peak parasitemia relative to mice that received serum from control mice (FIG. 2B). These data thus confirmed the results from the vaccinated μMT mice, indicating that neither B-cells nor antibody played a significant role in immunity induced by a chemically attenuated B. microti vaccine in this challenge model.

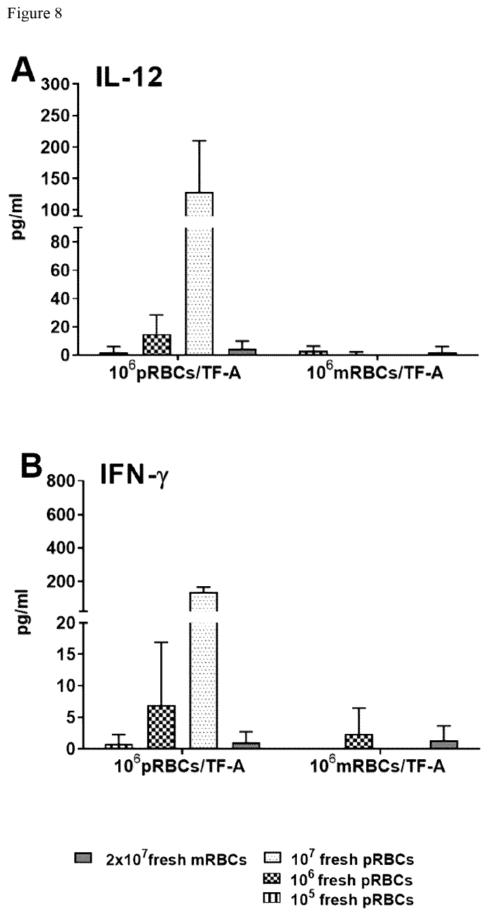

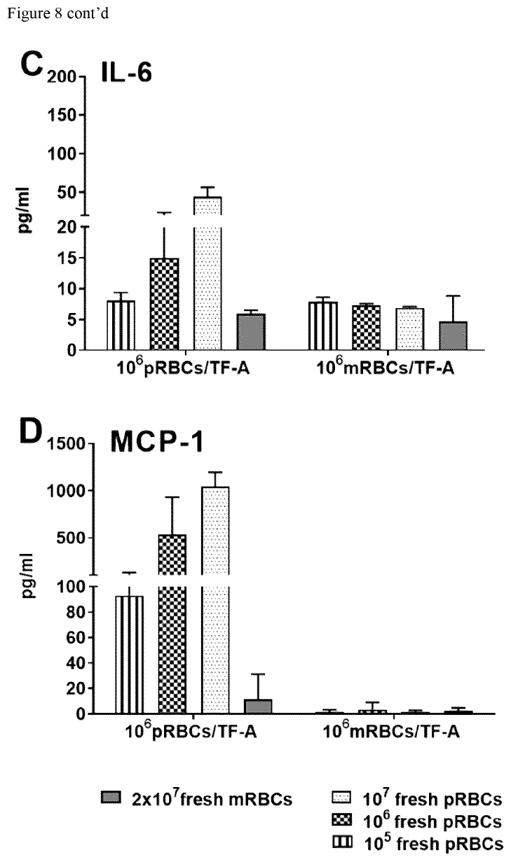

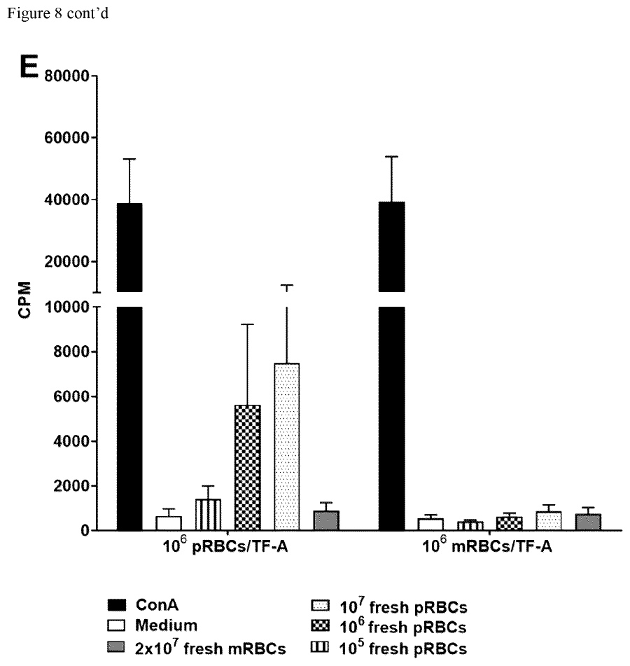

The inventors then investigated the role of T-cells in protection. Spleen cells from immunized mice proliferated significantly in vitro following stimulation with pRBCs and this response included production of IL-12p70, IFN-γ, IL-6 and MCP-1 in the culture supernatants (FIG. 8). TNF and IL-10 were also assessed but were undetectable. These cytokines were tested because of their known role in immunity to malaria (Low et al., 2018, Good and Stanisic, 2020) and because macrophages are known to play an important role in immunity to Babesia (Terkawi et al., 2015). We then depleted CD4+ and/or CD8⁺ T-cells from groups of immunized mice (n=5/group) using antibodies. Depletion of CD4⁺ T-cells or both CD4+ and CD8⁺ T-cells together, abrogated immunity, while depleting CD8⁺ T-cells alone had no effect (FIG. 2C). To examine the role of key cytokines/chemokines in protective immunity, mice were vaccinated, their cyto/chemokines depleted using antibodies specific for IL-12p40, IFN-γ or MCP-1, and then challenged. We observed a partial loss of protection following depletion of the macrophage chemoattractant protein, MCP-1, but no loss of protection following depletion of the other cytokines (FIG. 2D).

Given these data, it was asked whether macrophages were critical for immunity. Mice were vaccinated and clodronate liposomes then used to remove macrophages prior to challenge (Van Rooijen and Sanders, 1994). We observed a complete loss of immunity in vaccinated mice depleted of macrophages (FIG. 2E).

Homologous Protection can be Induced by a Lyophilized Whole Parasite B. microti Liposomal Vaccine

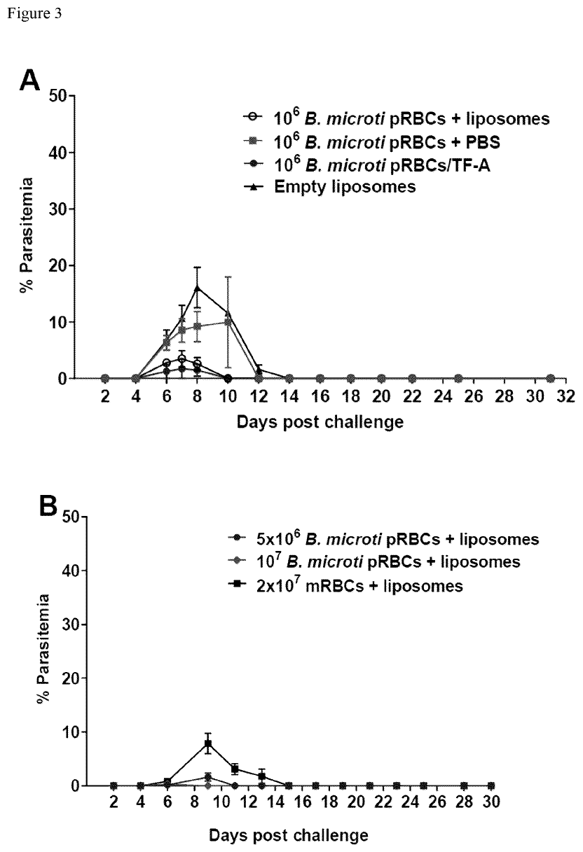

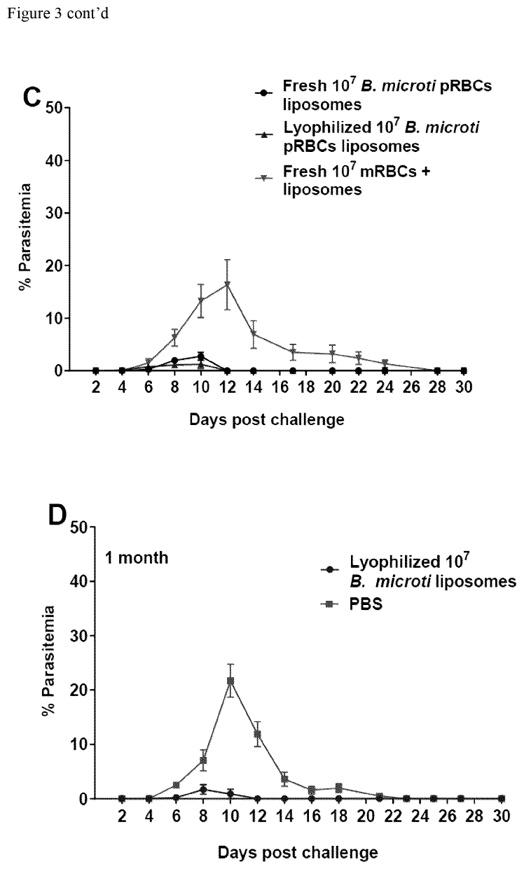

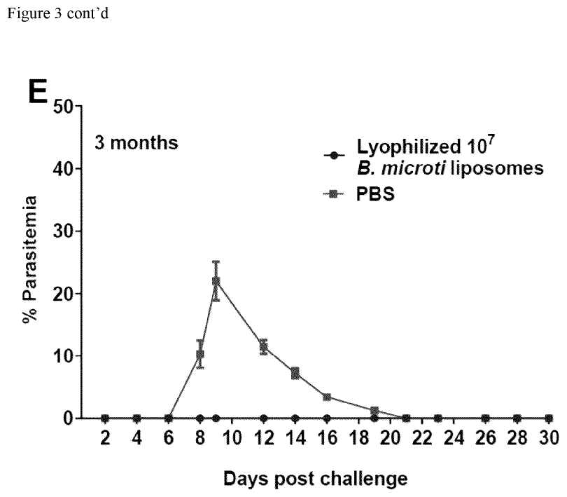

Because of the difficulties in cryopreserving a vaccine containing intact red cells, we asked whether the RBC membrane could be replaced with a lipid membrane with dead parasite encased within liposomes. Liposomes can be readily lyophilized (freeze-dried) and rehydrated without losing structure. We incorporated a mannosylated lipid core peptide, “F3” ((Giddam et al., 2016)), into the liposomes to facilitate targeting to APCs (FIG. 8A) and compared protective immunity induced by the chemically attenuated vaccine with that induced by the liposomal B. microti vaccine containing 10⁶ pRBCs. Peak parasitemias were not significantly different between these groups ([1.72%+1.72%] and [3.48%+1.42%], respectively) (FIG. 3A). The protective efficacy of the vaccine was dose-dependent, but liposomes containing either 5×10⁶ or 10⁷ pRBC induced significant protection as measured by peak parasitemia (FIG. 3B). We then assessed the impact of lyophilization on the efficacy of a vaccine containing 10⁷ B. microti pRBCs. Control groups were immunized with liposomes containing 10⁷ normal mouse red blood cells (mRBCs). The lyophilized vaccine gave equivalent protection to a fresh liposomal vaccine (FIG. 3C). Potency of the lyophilized vaccine, when stored at 4 C, was not diminished compared to fresh vaccine when administered 4 weeks, 6 weeks and 8 weeks after manufacture (first, second and third doses) (data not shown). Following vaccination, we observed that protection lasted at least as long as 3 months (FIG. 3D, E).

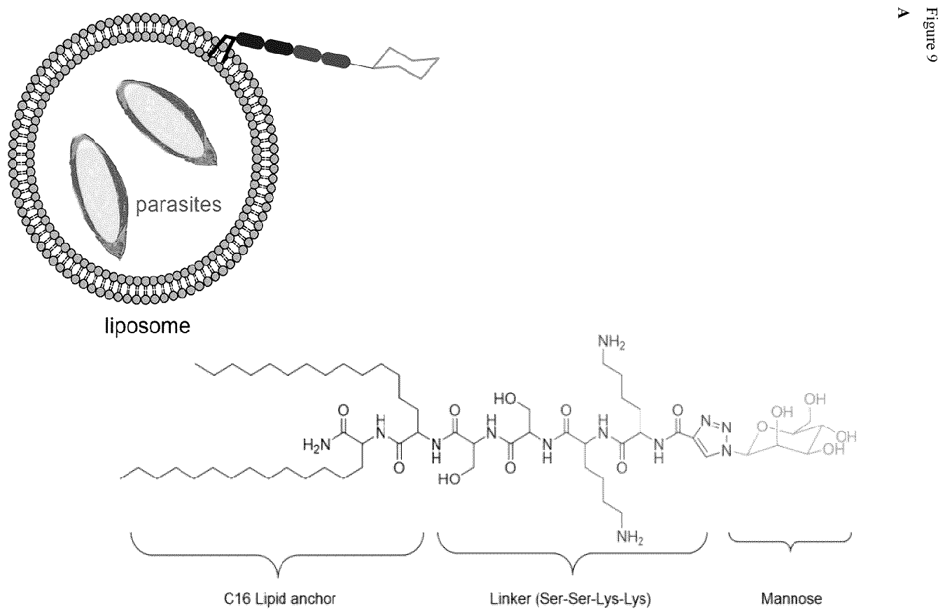

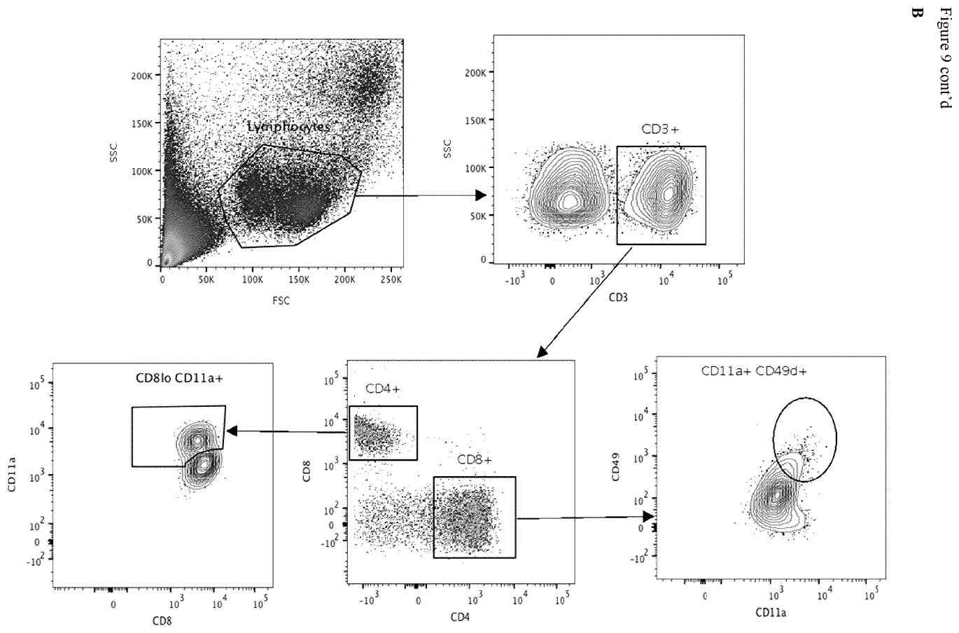

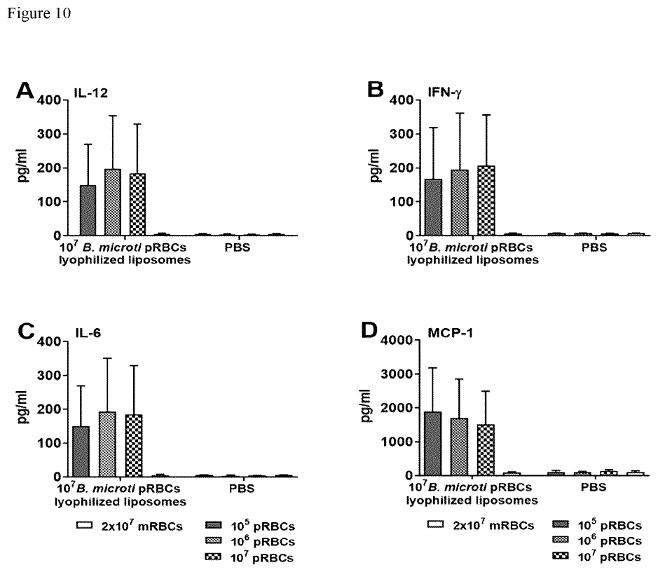

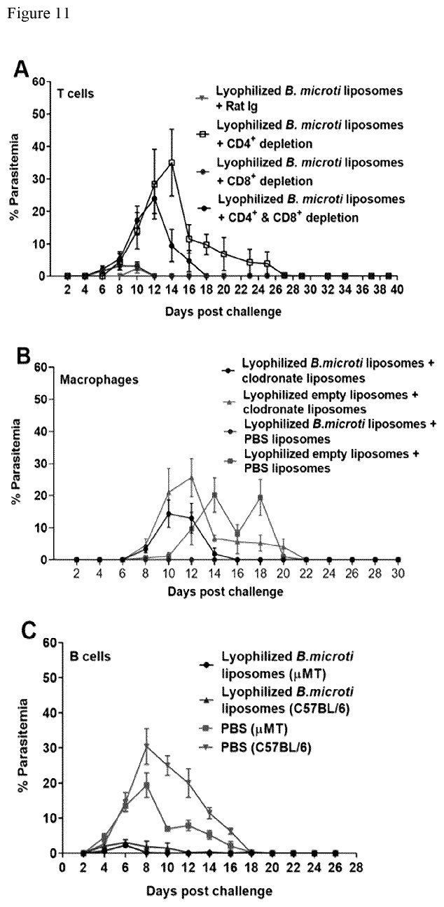

The inventors subsequently assessed both cellular and humoral immune responses (FIGS. 4A, B, C and D). Following vaccination, peripheral blood-derived CD4⁺ and CD8⁺ T-cells expressed activation markers ((CD11a^{hi}, CD49d^{hi}, CD8^{lo}, CD11a^{hi}, respectively) (FIG. 4A, B, FIG. 9). Spleen cells from vaccinated mice proliferated following stimulation with B. microti pRBC (FIG. 4C) and there was production of IL-12p70, IFN-γ, IL-6 and MCP-1 in the culture supernatant (FIG. 10). Serum from vaccinated mice contained low, but detectable, levels of antibodies to pRBC as detected by ELISA (FIG. 4D). Antibodies were noted just prior to the first boost of vaccine (day-13) and subsequently just pre-challenge (day-41). Similar to what we observed with the chemically attenuated vaccine, protection was sensitive to removal of CD4⁺ T-cells or macrophages prior to challenge and was apparent in mice lacking B-cells (FIGS. 11, A, B, and C). The liposomal vaccine thus presented similar characteristics to chemically attenuated parasites in terms of immune induction and protection from infection.

### Splenectomized Mice can be Protected by Vaccination

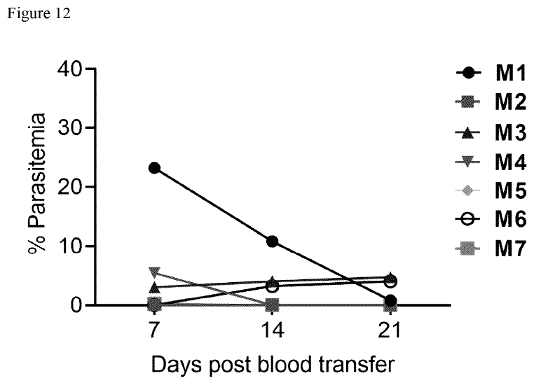

Severe babesiosis is more common in patients with generalized immunosuppression and asplenia (Mareedu et al., 2017). We thus asked whether mice that had been splenectomized could be protected by vaccination. BALB/c mice were splenectomized or sham-splenectomized and rested. They then received three doses of the lyophilized B. microti liposomal vaccine (or empty liposomes [as a control]) and were challenged two weeks after the final vaccination. Splenectomized mice that were given ‘empty’ liposomes had a peak parasitemia of 26.4%±9.35% whereas the peak parasitemia in splenectomized mice that were vaccinated reached 5.92±3.11% (p<0.05) (FIG. 4E). All splenectomized vaccinated mice resolved their microscopic parasitemia within 4 weeks of challenge, whereas mice that received the control vaccine had a microscopically patent infection for >60 days. To gauge the degree of protection, we transferred 100 μL of blood from vaccinated splenectomised mice six weeks after challenge. The recipient mice were then followed weekly for three weeks after receiving the blood to see if any developed a microscopic infection. We did not observe parasites in three of seven recipients (FIG. 12). While not demonstrating that any of the donor mice were completely free of parasites (as we only transferred 100 μL of blood), it is clear from these data that the parasite burdens of splenectomised vaccinated mice were either cleared or were very low.

Cross-Species Protection can be Induced by a Lyophilized Whole Parasite B. divergens Liposomal Vaccine

A vaccine for use in humans may not be made using mouse blood as the source of parasites. However, despite attempts to do so (Stahl, 2017), there are no reports of successful in vitro cultivation of B. microti in human red cells, of which we are aware. Therefore, to make a culture-derived vaccine for B. microti, we used a parasite that could be readily cultured in human blood, B. divergens (Grande et al., 1997), and asked whether a liposomal vaccine based on this parasite would provide heterologous protection against B. microti. B. divergens was cultured in vitro and a lyophilized vaccine was prepared containing 10⁷ B. divergens parasitized human RBC. Mice were given three doses of vaccine and control mice received either PBS or liposomes made with normal human red blood cells (hRBC). Following challenge with 1×10⁶ B. microti parasitized mouse red blood cells, vaccinated mice were strongly protected compared to both control groups (peak parasitemia of 2.69%+1.00% compared with 15.91%+4.90%, in a PBS group and 16.97%+5.60% in a control liposome group) (FIG. 4F). This cross-species protection was consistent with the splenocyte responses to B. microti and B. divergens. Splenocytes from mice vaccinated with B. divergens liposomes proliferated significantly in response to both B. divergens (compared to hRBCs) and to B. microti (compared to mRBCs) (FIG. 4G).

### DISCUSSION

The present examples a whole parasite vaccine for babesiosis that provides heterologous protection, can be lyophilized and re-hydrated prior to use and that can protect splenectomized animals. This study provides the rationale and pathway to human and animal vaccine trials.

This is not the first whole parasite vaccine for babesiosis; live attenuated calf-passaged vaccines have been developed for bovine babesiosis (Bock et al., 2004), but this is the first whole parasite vaccine described for human Babesia spp. parasites. Vaccines containing antigens derived from culture supernatants and adjuvanted with saponin were shown to have limited efficacy against B. bovis (in cattle) and against B. canis (in dogs) (Timms et al., 1983, Schetters et al., 2001). Subunit vaccine candidates have been described for human Babesia parasites but most required complete Freund's adjuvant (not suitable for human use) and induced limited protection (Munkhjargal et al., 2016, Terkawi et al., 2009, Man et al., 2017).

Where measured, protection induced by subunit vaccines correlated with the antibody response (Hadj-Kaddour et al., 2007). The need for potent adjuvants to induce the high levels of antibody required for protection together with antigenic polymorphism of surface proteins represent major challenges for subunit vaccines. Similar challenges exist for malaria vaccine development and these are yet to be overcome (Crompton et al., 2010, Good and Stanisic, 2020). However, the subunit vaccine approach is fundamentally different to the whole parasite approach where the ability to present all antigens of the parasite significantly reduces the challenges presented by antigenic polymorphism, as has been shown for malaria (Good et al., 2013). Furthermore, subunit vaccines rely on induction of antibodies to block merozoite invasion whereas the whole parasite vaccines described here act independently of antibody but require effector CD4⁺ T-cells and macrophages. Because T-cells recognize processed antigens, targets need not be surface antigens. Intracellular ‘house-keeping’ antigens, such as enzymes can be targets of T cells. Whole parasite vaccines for malaria blood-stage parasites have now entered clinical trials (Stanisic et al., 2018), and there is already significant progress in whole parasite vaccines for the sporozoite stage of malaria (Seder et al., 2013, Mordmuller et al., 2017). As the only other intra-erythrocytic parasite of humans, there is an opportunity to build on the substantial knowledge garnered for malaria.

It was observed that mice lacking B-cells could be protected following vaccination as well as normal mice. This finding was surprising; however, we had shown that serum from mice vaccinated and protected by some whole parasite malaria vaccines did not contain protective antibodies (Good et al., 2013). Our data further showed that protection was ablated by removal of CD4⁺ T-cells prior to challenge. How the whole parasite vaccines described here activate CD4⁺ T-cells in the absence of an adjuvant is not fully understood. The inventors observed that lysed infected red cells do not induce immunity, suggesting that either the red cell membrane or the liposomal membrane are critical. In this model, parasite antigens encased within a membrane would closely mimic natural infection, which is known to induce strong immunity in rodent models (Cox and Young, 1969). Data from whole parasite vaccines in malaria show that the importance of the membrane is to target the cargo of parasite antigens to the antigen presenting cells in both the spleen and liver where immune responses are initiated (Good et al., 2013, Giddam et al., 2016). It is possible that the same mechanism of immune induction applies to Babesia whole parasite vaccines, but that the ability to immunize splenectomized animals indicates that splenic APCs are not essential and that APCs in tissues other than the spleen can initiate broad ranging CD4⁺ T-cell immune responses. RBC membrane-expressed parasite antigens are likely to provide the mechanism of targeting the attenuated vaccine to APCs while the synthetic mannose added to the liposomes will aid targeting of the liposomal vaccine (Giddam et al., 2016). Parasite antigens are likely to be embedded in the liposome membrane and may also aid targeting to APCs.

The present data suggest that downstream activation of macrophages is important. Macrophages are known major players in innate immunity and once activated recognize various microbial products as well as other ‘danger’ signals (Gasteiger et al., 2017). Macrophages are known to play an important role in natural resistance to Babesia (Terkawi et al., 2015) and in non-opsonic phagocytosis in malaria (Chua et al., 2013). The macrophage depletion studies support this as the likely mechanism for whole parasite vaccine-mediated immunity in Babesia. Prolonged protection was observed following vaccination with both the chemically attenuated and liposomal vaccine. Persisting antigen, as either a depot or in a different form, may contribute to this prolonged protection (Woodland and Kohlmeier, 2009).

The present example highlights the ability of B. divergens to grow to a parasitemia of over 40% in human RBCs. As such, one hundred millilitres of human blood may be sufficient to generate ˜20,000 doses of vaccine, assuming 10⁷ pRBC equivalents per dose.

In conclusion, the present example has provided valuable data regarding vaccine characteristics, homologous and heterologous efficacy, dose-responsiveness, immune mechanisms of protection, and short-term stability for a Babesia vaccine.

### REFERENCES

ACOSTA, M. E., ENDER, P. T., SMITH, E. M. & JAHRE, J. A. 2013. Babesia microti infection, eastern Pennsylvania, USA. Emerg Infect Dis, 19, 1105-7.
ALVARADO-RYBAK, M., SOLANO-GALLEGO, L. & MILLAN, J. 2016. A review of piroplasmid infections in wild carnivores worldwide: importance for domestic animal health and wildlife conservation. Parasit Vectors, 9, 538.
BOCK, R., JACKSON, L., DE VOS, A. & JORGENSEN, W. 2004. Babesiosis of cattle. Parasitology, 129 Suppl, S247-69.
CALLOW, L. & MELLORS, L. 1966. A NEW VACCINE FOR BABESIA ARGENTINA INFECTION PREPARED IN SPLENECTOMISED CALVES. Australian Veterinary Journal, 42, 464-465.
CARCY, B., PRECIGOUT, E., SCHETTERS, T. & GORENFLOT, A. 2006. Genetic basis for GPI-anchor merozoite surface antigen polymorphism of Babesia and resulting antigenic diversity. Vet Parasitol, 138, 33-49.
CDC 2012. Babesiosis surveillance—18 states, 2011. Morbidity and Mortality Weekly Report, 61, 505: Centers for Disease Control.
CHUA, C. L., BROWN, G., HAMILTON, J. A., ROGERSON, S. & BOEUF, P. 2013. Monocytes and macrophages in malaria: protection or pathology? Trends Parasitol, 29, 26-34.
COX, F. & YOUNG, A. 1969. Acquired immunity to Babesia microti and Babesia rodhaini in mice. Parasitology, 59, 257-268.
CROMPTON, P. D., PIERCE, S. K. & MILLER, L. H. 2010. Advances and challenges in malaria vaccine development. J Clin Invest, 120, 4168-78.
FANG, D. C. & MCCULLOUGH, J. 2016. Transfusion-Transmitted Babesia microti. Transfus Med Rev, 30, 132-8.
GASTEIGER, G., D'OSUALDO, A., SCHUBERT, D. A., WEBER, A., BRUSCIA, E. M. & HARTL, D. 2017. Cellular Innate Immunity: An Old Game with New Players. J Innate Immun, 9, 111-125.
GIDDAM, A. K., REIMAN, J. M., ZAMAN, M., SKWARCZYNSKI, M., TOTH, I. & GOOD, M. F. 2016. A semi-synthetic whole parasite vaccine designed to protect against blood stage malaria. Acta Biomater, 44, 295-303.
GOOD, M. F., REIMAN, J. M., RODRIGUEZ, I. B., ITO, K., YANOW, S. K., EL-DEEB, I. M., BATZLOFF, M. R., STANISIC, D. I., ENGWERDA, C., SPITHILL, T., HOFFMAN, S. L., LEE, M. & MCPHUN, V. 2013. Cross-species malaria immunity induced by chemically attenuated parasites. J Clin Invest.
GOOD, M. F. & STANISIC, D. I. 2020. Whole parasite vaccines for the asexual blood stages of Plasmodium. Immunol Rev, 293, 270-282.
GRANDE, N., PRECIGOUT, E., ANCELIN, M. L., MOUBRI, K., CARCY, B., LEMESRE, J. L., VIAL, H. & GORENFLOT, A. 1997. Continuous in vitro culture of Babesia divergens in a serum-free medium. Parasitology, 115 (Pt 1), 81-9.
GRAY, J., ZINTL, A., HILDEBRANDT, A., HUNFELD, K. P. & WEISS, L. 2010. Zoonotic babesiosis: overview of the disease and novel aspects of pathogen identity. Ticks Tick Borne Dis, 1, 3-10.
HADJ-KADDOUR, K., CARCY, B., VALLET, A., RANDAZZO, S., DELBECQ, S., KLEUSKENS, J., SCHETTERS, T., GORENFLOT, A. & PRECIGOUT, E. 2007. Recombinant protein Bd37 protected gerbils against heterologous challenges with isolates of Babesia divergens polymorphic for the bd37 gene. Parasitology, 134, 187-196
HERWALDT, B. L., LINDEN, J. V., BOSSERMAN, E., YOUNG, C., OLKOWSKA, D. & WILSON, M. 2011. Transfusion-associated babesiosis in the United States: a description of cases. Ann Intern Med, 155, 509-19.
KRAUSE, P. J. 2019. Human babesiosis. Int J Parasitol, 49, 165-174.
LEMIEUX, J. E., TRAN, A. D., FREIMARK, L., SCHAFFNER, S. F., GOETHERT, H., ANDERSEN, K. G., BAZNER, S., LI, A., MCGRATH, G., SLOAN, L., VANNIER, E., MILNER, D., PRITT, B., ROSENBERG, E., TELFORD, S., 3RD, BAILEY, J. A. & SABETI, P. C. 2016. A global map of genetic diversity in Babesia microti reveals strong population structure and identifies variants associated with clinical relapse. Nat Microbiol, 1, 16079.
LOW, L. M., SSEMAGANDA, A., LIU, X. Q., HO, M. F., OZBERK, V., FINK, J., SUNDAC, L., ALCORN, K., MORRISON, A., O′CALLAGHAN, K., GERRARD, J., STANISIC, D. I. & GOOD, M. F. 2018. Controlled infection immunization using delayed death drug treatment elicits protective immune responses to blood-stage malaria parasites. Infect Immun.
MAKOBONGO, M. O., RIDING, G., XU, H., HIRUNPETCHARAT, C., KEOUGH, D., DE JERSEY, J., WILLADSEN, P. & GOOD, M. F. 2003. The purine salvage enzyme hypoxanthine guanine xanthine phosphoribosyl transferase is a major target antigen for cell-mediated immunity to malaria. Proc Natl Acad Sci USA, 100, 2628-33.
MAN, S., FU, Y., GUAN, Y., FENG, M., QIAO, K., LI, X., GAO, H. & CHENG, X. 2017. Evaluation of a Major Surface Antigen of Babesia microti Merozoites as a Vaccine Candidate against Babesia Infection. Front Microbiol, 8, 2545.
MAREEDU, N., SCHOTTHOEFER, A. M., TOMPKINS, J., HALL, M. C., FRITSCHE, T. R. & FROST, H. M. 2017. Risk factors for severe infection, hospitalization, and prolonged antimicrobial therapy in patients with babesiosis. The American Journal of Tropical Medicine and Hygiene, 97, 1218-1225.
MORDMULLER, B., SURAT, G., LAGLER, H., CHAKRAVARTY, S., ISHIZUKA, A. S., LALREMRUATA, A., GMEINER, M., CAMPO, J. J., ESEN, M., RUBEN, A. J., HELD, J., CALLE, C. L., MENGUE, J. B., GEBRU, T., IBANEZ, J., SULYOK, M., JAMES, E. R., BILLINGSLEY, P. F., NATASHA, K. C., MANOJ, A., MURSHEDKAR, T., GUNASEKERA, A., EAPPEN, A. G., LI, T., STAFFORD, R. E., LI, M., FELGNER, P. L., SEDER, R. A., RICHIE, T. L., SIM, B. K., HOFFMAN, S. L. & KREMSNER, P. G. 2017. Sterile protection against human malaria by chemoattenuated PfSPZ vaccine. Nature, 542, 445-449.
MUNKHJARGAL, T., YOKOYAMA, N. & IGARASHI, I. 2016. Recombinant methionine aminopeptidase protein of Babesia microti: immunobiochemical characterization as a vaccine candidate against human babesiosis. Parasitol Res, 115, 3669-76.
PINZON-CHARRY, A., MCPHUN, V., KIENZLE, V., HIRUNPETCHARAT, C., ENGWERDA, C., MCCARTHY, J. & GOOD, M. F. 2010. Low doses of killed parasite in CpG elicit vigorous CD4+ T cell responses against blood-stage malaria in mice. J Clin Invest, 120, 2967-78.
RAJA, A. I., CAI, Y., REIMAN, J. M., GROVES, P., CHAKRAVARTY, S., MCPHUN, V., DOOLAN, D. L., COCKBURN, I., HOFFMAN, S. L., STANISIC, D. I. & GOOD, M. F. 2016. Chemically Attenuated Blood-Stage Plasmodium yoelii Parasites Induce Long-Lived and Strain-Transcending Protection. Infect Immun, 84, 2274-2288.
REIMAN, J. M., KUMAR, S., RODRIGUEZ, I. B., GNIDEHOU, S., ITO, K., STANISIC, D. I., LEE, M., MCPHUN, V., MAJAM, V., WILLEMSEN, N. M., BATZLOFF, M. R., RAJA, A. I., DOOLEY, B., HOFFMAN, S. L., YANOW, S. K. & GOOD, M. F. 2018. Induction of immunity following vaccination with a chemically attenuated malaria vaccine correlates with persistent antigenic stimulation. Clin Transl Immunology, 7, e1015.
RODRIGUEZ, M., ALHASSAN, A., ORD, R. L., CURSINO-SANTOS, J. R., SINGH, M., GRAY, J. & LOBO, C. A. 2014. Identification and characterization of the RouenBd1987 Babesia divergens Rhopty-Associated Protein 1. PLOS One, 9, e107727. SCHETTERS, T. P., KLEUSKENS, J., SCHOLTES, N., GORENFLOT, A., MOUBRI, K. & VERMEULEN, A. 2001. Vaccination of dogs against heterologous Babesia canis infection using antigens from culture supernatants. Veterinary Parasitology, 100, 75-86.
SCHNITTGER, L., RODRIGUEZ, A. E., FLORIN-CHRISTENSEN, M. & MORRISON, D. A. 2012. Babesia: a world emerging. Infection, Genetics and Evolution, 12, 1788-1809.
SEDER, R. A., CHANG, L. J., ENAMA, M. E., ZEPHIR, K. L., SARWAR, U. N., GORDON, I. J., HOLMAN, L. A., JAMES, E. R., BILLINGSLEY, P. F., GUNASEKERA, A., RICHMAN, A., CHAKRAVARTY, S., MANOJ, A., VELMURUGAN, S., LI, M. L., RUBEN, A. J., LI, T., EAPPEN, A. G., STAFFORD, R. E., PLUMMER, S. H., HENDEL, C. S., NOVIK, L., COSTNER, P. J. M., MENDOZA, F. H., SAUNDERS, J. G., NASON, M. C., RICHARDSON, J. H., MURPHY, J., DAVIDSON, S. A., RICHIE, T. L., SEDEGAH, M., SUTAMIHARDJA, A., FAHLE, G. A., LYKE, K. E., LAURENS, M. B., ROEDERER, M., TEWARI, K., EPSTEIN, J. E., SIM, B. K. L., LEDGERWOOD, J. E., GRAHAM, B. S., HOFFMAN, S. L. & TEAM, V. S. 2013. Protection Against Malaria by Intravenous Immunization with a Nonreplicating Sporozoite Vaccine. Science, 341, 1359-1365.
SSEMAGANDA, A., GIDDAM, A. K., LOW, L. M., LIU, X. Q., HO, M.-F., ZAMAN, M., HUSSEIN, W. M., SKWARCZYNSKI, M., TOTH, I. & STANISIC, D. I. 2020. Mannosylated liposomes formulated with whole parasite P. falciparum blood-stage antigens are highly immunogenic in mice. Vaccine, 38, 1494-1504.
STAHL, P., POINSIGNON, M. D., ET AL 2017. Case report of the patient source of the Babesia microti R1 reference strain and implications for travelers. Journal of Travel Medicine, 25, 1-8.
STANISIC, D. I., FINK, J., MAYER, J., COGHILL, S., GORE, L., LIU, X. Q., EL-DEEB, I., RODRIGUEZ, I. B., POWELL, J., WILLEMSEN, N. M., DE, S. L., HO, M. F., HOFFMAN, S. L., GERRARD, J. & GOOD, M. F. 2018. Vaccination with chemically attenuated Plasmodium falciparum asexual blood-stage parasites induces parasite-specific cellular immune responses in malaria-naive volunteers: a pilot study. BMC Med, 16, 184.
STEINHARDT, L. C., RICHIE, T. L., YEGO, R., AKACH, D., HAMEL, M. J., GUTMAN, J. R., WIEGAND, R. E., NZUU, E. L., DUNGANI, A., KC, N., MURSHEDKAR, T., CHURCH, L. W. P., SIM, B. K. L., BILLINGSLEY, P. F., JAMES, E. R., ABEBE, Y., KARIUKI, S., SAMUELS, A. M., OTIENO, K., SANG, T., KACHUR, S. P., STYERS, D., SCHLESSMAN, K., ABARBANELL, G., HOFFMAN, S. L., SEDER, R. A. & ONEKO, M. 2020. Safety, Tolerability, and Immunogenicity of Plasmodium falciparum Sporozoite Vaccine Administered by Direct Venous Inoculation to Infants and Young Children: Findings From an Age De-escalation, Dose-Escalation, Double-blind, Randomized Controlled Study in Western Kenya. Clin Infect Dis, 71, 1063-1071.
TERKAWI, M. A., ABOGE, G., JIA, H., GOO, Y. K., OOKA, H., YAMAGISHI, J., NISHIKAWA, Y., YOKOYAMA, N., IGARASHI, I., KAWAZU, S. I., FUJISAKI, K. & XUAN, X. 2009. Molecular and immunological characterization of Babesia gibsoni and Babesia microti heat shock protein-70. Parasite Immunol, 31, 328-40.
TERKAWI, M. A., CAO, S., HERBAS, M. S., NISHIMURA, M., LI, Y., MOUMOUNI, P. F., PYAROKHIL, A. H., KONDOH, D., KITAMURA, N., NISHIKAWA, Y., KATO, K., YOKOYAMA, N., ZHOU, J., SUZUKI, H., IGARASHI, I. & XUAN, X. 2015. Macrophages are the determinant of resistance to and outcome of nonlethal Babesia microti infection in mice. Infect Immun, 83, 8-16. TIMMS, P., DALGLIESH, R., BARRY, D., DIMMOCK, C. & RODWELL, B. 1983. Babesia bovis: comparison of culture-derived parasites, non-living antigen and conventional vaccine in the protection of cattle against heterologous challenge. Australian Veterinary Journal, 60, 75-77.
TONNETTI, L., TOWNSEND, R. L., DEISTING, B. M., HAYNES, J. M., DODD, R. Y. & STRAMER, S. L. 2019. The impact of Babesia microti blood donation screening. Transfusion, 59, 593-600.
VAN ROOIJEN, N. & SANDERS, A. 1994. Liposome mediated depletion of macrophages: mechanism of action, preparation of liposomes and applications. J Immunol Methods, 174, 83-93.
VANNIER, E. G., DIUK-WASSER, M. A., BEN MAMOUN, C. & KRAUSE, P. J. 2015. Babesiosis. Infect Dis Clin North Am, 29, 357-70.
VERMA, N., PURI, A., ESSUMAN, E., SKELTON, R., ANANTHARAMAN, V., ZHENG, H., WHITE, S., GUNALAN, K., TAKEDA, K., BAJPAI, S., LEPORE, T. J., KRAUSE, P. J., ARAVIND, L. & KUMAR, S. 2020. Antigen Discovery, Bioinformatics and Biological Characterization of Novel Immunodominant Babesia microti Antigens. Sci Rep, 10, 9598.
WANG, J., YANG, J., GAO, S., WANG, X., SUN, H., LV, Z., LI, Y., LIU, A., LIU, J., LUO, J., GUAN, G. & YIN, H. 2020. Genetic Diversity of Babesia bovis MSA-1, MSA-2b and MSA-2c in China. Pathogens, 9.
WOODLAND, D. L. & KOHLMEIER, J. E. 2009. Migration, maintenance and recall of memory T cells in peripheral tissues. Nat Rev Immunol, 9, 153-61.

### Example 2—Testing of a Vaccine Based on B. divergens and Human RBCs in a Canine Model of Babesia (Babesia Rossi)

B. divergens will be cultured in vitro in human red blood cells according to established methods to obtain parasites for the vaccine. The B. divergens parasitised red blood cells (pRBC) will be subjected to 5-6 cycles of freeze-thawing. Liposomes will be produced using the thin film hydration method and will be formulated with the lysed B. divergens pRBCs. Empty liposomes will be prepared as a control. Liposomal formulations will be freeze-dried to produce a lyophilised product which will be reconstituted in saline-for-injection immediately prior to administration.

Groups of 3-4 beagles will be immunised with liposomes containing different doses of lysed B. divergens pRBCs. One group of beagles will receive empty liposomes as a control group. Each beagle will receive 3 vaccine doses, 2 weeks apart. Two weeks after the final vaccine dose, the animals will be challenged with live B. rossi pRBC derived from a donor beagle. Clinical outcomes and parasitemias will be assessed in the vaccinated, challenged animals. Immunogenicity will also be evaluated.

1. An immunogenic composition for administration to a first mammal, said composition comprising erythrocytes and an erythrocytic organism, wherein the erythrocytes are from a second mammal of a species different from the first mammal.

2. The immunogenic composition of claim 1, wherein the erythrocytic organism is attenuated, inactivated and/or killed.

3. The immunogenic composition of claim 1 or claim 2, wherein the erythrocytes have been previously infected with the erythrocytic organism.

4. The immunogenic composition of any one of the preceding claims, which does not comprise or is substantially free of an adjuvant.

5. The immunogenic composition of any one of claims 1 to 3, wherein the immunogenic composition further comprises an adjuvant.

6. The immunogenic composition of claim 5, wherein the adjuvant is or comprises a lipid-based adjuvant.

7. The immunogenic composition of any one of the preceding claims, wherein the erythrocytes are contained in or otherwise associated with a particle.

8. The immunogenic composition of claim 7, wherein the particle is a lipid-based particle.

9. The immunogenic composition of any one of the preceding claims, wherein the erythrocytes are contained in or otherwise associated with a liposome.

10. The immunogenic composition of any one of the preceding claims, further comprising a cell targeting ligand.

11. The immunogenic composition of any one of the preceding claims, wherein the erythrocytic organism has been chemically attenuated or inactivated.

12. The immunogenic composition of claim 11, wherein the erythrocytic organism has been chemically attenuated with a DNA binding agent.

13. The immunogenic composition of claim 12, wherein the DNA binding agent is selected from centanamycin, tafuramycin A and any combination thereof.

14. The immunogenic composition of any one of claims 1 to 11, wherein the erythrocytes have been treated to inactivate or kill the erythrocytic organism.

15. The immunogenic composition of any one of the preceding claims, wherein the erythrocytes are intact.

16. The immunogenic composition of any one of claims 1 to 14, wherein the erythrocytes are lysed.

17. The immunogenic composition of any one of the preceding claims, wherein the first mammal is a non-human animal.

18. The immunogenic composition of claim 17, wherein the first mammal is canine or bovine.

19. The immunogenic composition of any one of the preceding claims, wherein the second mammal is human.

20. The immunogenic composition of any one of the preceding claims, wherein the erythrocytic organism is an intra-erythrocytic organism or an intra-erythrocytic parasite.

21. The immunogenic composition of any one of the preceding claims, wherein the erythrocytic organism is selected from the group consisting of a Babesia sp., an Anaplasma sp., an Ehrlichia sp., a Trypanosoma sp., a Theileria sp., a Hepatozoon sp., a Mycoplasma sp., a Bartonella sp. and any combination thereof.

22. The immunogenic composition of claim 21, wherein the erythrocytic organism is or comprises a Babesia sp.

23. The immunogenic composition of claim 22, wherein the erythrocytic organism is selected from the group consisting of B. bigemina, B. bovis, B. caballi, B. canis, B. divergens, B. rossi, B. microti, B motasi, and any combination thereof.

24. The immunogenic composition of any one of claims 21 to 23, wherein the erythrocytic organism is or comprises a Babesia sp. and an Anaplasma sp.

25. The immunogenic composition of any one of claims 21 to 24, wherein the erythrocytic organism is or comprises Babesia divergens.

26. The immunogenic composition of any one of the preceding claims, wherein the immunogenic composition provides heterologous protection against an infection, disease or condition associated with one or more other isolates, strains and/or species of the erythrocytic organism.

27. The immunogenic composition of claim 26, wherein the immunogenic composition provides heterologous protection against an infection, disease or condition associated with one or more other isolates, strains and/or species of Babesia.

28. The immunogenic composition of any one of the preceding claims, wherein the immunogenic composition is for use in a method of:
(a) eliciting an immune response in the first mammal; and/or
(b) preventing, treating or ameliorating an infection, disease or condition associated with the erythrocytic organism in the first mammal.

29. A method of preventing, treating or ameliorating an infection, disease or condition associated with an erythrocytic organism in a first mammal, said method including the step of administering to the first mammal a therapeutically effective amount of the immunogenic composition of any one of claims 1 to 28.

30. A method of inducing an immune response in a first mammal, said method including the step of administering to the first mammal, an effective amount of the immunogenic composition of any one of claims 1 to 28.

31. Use of the immunogenic composition of any one of claims 1 to 28 in the manufacture of a medicament for:
(a) eliciting an immune response in the first mammal; and/or
(b) preventing, treating or ameliorating an infection, disease or condition associated with the erythrocytic organism in the first mammal.
